(19) United States
(12) Patent Application Publication
(10) Pub. No.: US 2018/0172686 A1
(43) Pub. Date: Jun. 21, 2018

(54) Method of Assessing Infection Status

(71) Applicant: MEDELA HOLDING AG, Baar (CH)

(72) Inventor: Foteini Kakulas, Bullsbrook (AU)

(73) Assignee: MEDELA HOLDING AG, Baar (CH)

(21) Appl. No.: 15/847,398

(22) Filed: Dec. 19, 2017

Related U.S. Application Data
(63) Continuation of application No. 13/859,438, filed on Apr. 9, 2013, now abandoned.
(60) Provisional application No. 61/621,709, filed on Apr. 9, 2012.

(51) Int. Cl. G01N 33/569; G01N 33/68

(52) U.S. Cl. CPC G01N 33/56972; G01N 33/6863; G01N 2800/38; G01N 33/6854; G01N 33/569

(57) ABSTRACT

The present invention relates to a method for assessing the infection status of a subject and in particular to a method for assessing the infection status of a subject by analysing the cellular and/or humoral composition of breastmilk from said subject. The invention has been developed primarily for use as a method for detecting infection in a breastfeeding mother and/or infant.

This application claims the benefit of U.S. Provisional Application Ser. No. 61/621,709, filed Apr. 9, 2012, the disclosure of which is incorporated by reference herein.

### FIELD OF THE INVENTION

The present invention relates to a method for assessing the infection status of a subject and in particular to a method for assessing the infection status of a subject by analysing the cellular and/or humoral composition of breastmilk from said subject. The invention has been developed primarily for use as a method for detecting infection in a breastfeeding mother and/or infant and will be described hereinafter with reference to this application. However, it will be appreciated that the invention is not limited to this particular field of use.

### BACKGROUND

Any discussion of the background art throughout the specification should in no way be considered as an admission that such art is widely known or forms part of common general knowledge in the field.

Breastmilk is an excellent source of macro- and micro-nutrients that ensure normal development of the human infant at the early stages of life (Lawrence and Lawrence, 1999). In addition to being nutritious, breastmilk contains a plethora of bioactive molecules which confer short- and long-term benefits to the neonate (Lönnerdal, 2003; Le Huërou-Luron et al., 2010; Savino et al., 2010), as well as maternal cells of various types, the role of which during breastfeeding is not fully established.

Evidence has been accumulating providing support for positive long-term effects associated with increased duration of breastfeeding, such as improved cognitive ability, enhanced mucosal and immune system development, and reduced incidence of inflammatory bowel disease, atopic disease, hypertension, type 2 diabetes and obesity later in life (Oddy et al., 1999; Horta et al., 2007; Kramer, 2010). Short-term benefits of breastfeeding have been studied more frequently, and are associated with self-regulation of energy intake (Dewey and Lönnerdal, 1986; Taveras et al., 2004), lower risk of necrotising enterocolitis in pre-term infants, and reduced susceptibility to gastrointestinal and respiratory infections (Howie et al., 1990; COsar et al., 1999; Kramer, 2010; Le Huërou-Luron et al., 2010). And, although protective effects of breastfeeding against infection have been observed both in developing and developed countries (Kramer, 2010), the underlying mechanisms through which they are conferred are still the subject of ongoing research.

Through breastfeeding, the transfer of immune factors from the mother to the infant, which had started already in utero, continues postnatally (Zhou et al., 2000; Perez et al., 2006). This not only protects the neonate from infection, but it also assists in the development of the intestinal mucosa and the infant's own defences (Walker, 2004; Le Huërou-Luron et al., 2010). The importance of this postnatal regulation and protection through breastmilk is evidenced by the different intestinal colonisation and greater disease susceptibility of the formula-fed compared to breastfed infants (Hanson and Winberg, 1972; Falk et al., 1998; Wright et al., 1998; Perez et al., 2006). The immune factors transferred to the infant through breastmilk include both maternal leukocytes and bioactive molecules with antimicrobial, anti-inflammatory, antioxidant and prebiotic activities (Smith and Goldman, 1968; Kmetz et al., 1970; Weiler et al., 1983; Jain et al., 1989; Zhou et al., 2000; Lönnerdal, 2003; Kourtis et al., 2007; Bryan et al., 2007).

In addition to being a well-balanced source of amino acids, specifically serving the growth needs of the human infant, some breastmilk proteins assist in the digestion and absorption of micro- and macro-nutrients (amylase, β-casein, lactoferrin, haptocorrin, α₁-antitrypsin), while others exert antimicrobial and immunomodulatory activities (Lönnerdal, 2003). Amongst those, κ-casein, lysozyme, lactoferrin, haptocorrin, α-lactalbumin, lactoperoxidase, cytokines and immunoglobulins (Ig), being resistant to proteolysis, enhance the breastfed infant's defence against pathogens, often acting synergistically (Lönnerdal, 2003). Secretory IgA (sIgA), the major immunoglobulin type in human milk, confers maternal acquired immunity to the infant at a time when its immune system is immature (Telemo et al., 1996). A number of immunomodulatory cytokines have been detected in breastmilk (Grosvenor et al., 1993) with either inflammatory or anti-inflammatory properties (Buescher, 2001; Lönnerdal, 2003). Antimicrobial, immunomodulatory and prebiotic effects similar to breastmilk proteins are also conferred by breastmilk oligosaccharides (Newburg, 1997; Coppa et al., 2004), some of which mimic cell surface markers, influencing cell-to-cell communication in the neonate gut (Bode et al., 2004). Vitamins (A, C and E) and other molecules with antioxidant properties (catalase, glutathione peroxidase, superoxide dismutase) are also present in breastmilk, enhancing the antioxidant defences of the neonate, which are also activated in incidents of infection (Goldman et al., 1990; Friel et al., 2002). Other soluble factors with immunomodulatory and protective properties have also been discovered in breastmilk more recently (Vidal et al., 2001).

The immunoreactive biochemical factors of human breastmilk are complemented by maternal breastmilk leukocytes, which have also been hypothesised to confer active immunity to the infant as well as protect the mammary gland from infection (Hanson and Winberg, 1972; Jain et al., 1989; Wirt et al., 1992; Zhou et al., 2000; Labéta et al., 2000; Lönnerdal, 2003). As early as 1838, Donné performed microscopic examinations of human colostrum, followed by Henle (1841). Both scientists noted for the first time the presence of cells in colostrum, then named “granular bodies” (Donné, 1838) or “colostrum bodies” (Henle, 1841). Today we know that both colostrum and mature breastmilk contain cells of various types, from epithelial to stem-like cells to leukocytes (Cregan et al., 2007). Some of these are endogenous to the mammary gland, whilst others, such as the leukocytes, migrate to this site via the lymphatic vessels and systemic circulation (Roux et al., 1977; Goldman and Goldblum, 1997). The leukocytes can comprise granulocytes, B and T lymphocytes, monocytes and macrophages, and have been thought to constitute an important part of the cellular portion of colostrum, decreasing in mature breastmilk.

Noticeably, the concentrations of immune factors in breastmilk are by no means stable. Breastmilk is a complex and very dynamic fluid, with a changing composition in response to the stage of lactation (Lönnerdal, 2003; Le Huërou-Luron et al., 2010) as well as the health condition of the mother and/or the breastfed infant (Kourtis et al., 2007; Bryan et al., 2007). For example, as lactation progresses the protein content of breastmilk decreases (Lönnerdal et al., 1976; Kunz and Lönnerdal, 1992). Other bioactive components are altered soon after birth, with distinct differences found between colostrum and mature breastmilk, such as those of casein, which is virtually undetectable in colostrum (Kunz and Lönnerdal, 1990; 1992), sIgA, which is found at very high concentrations in colostrum, decreasing to about half in mature breastmilk (Goldman, 1993), and lactoferrin, which decreases about seven times from colostrum to mature breastmilk (Bernt and Walker, 2001). In addition to bioactive molecules, there is a marked difference in the leukocyte content between colostrum and mature breastmilk.

In light of the above, it will be appreciated that breastmilk is a highly complex substance and it is noteworthy that during lactation, approximately one third of the mother's daily energy intake will be utilised for milk production. As such, the mother's metabolism is significantly altered to produce breastmilk containing the array of proteins mentioned above, some of which play major physiological roles and are uniquely produced in the lactating breast.

Midwifes and lactation consultants provide invaluable services to breastfeeding mothers, however, deterioration of a mother's infection status may lead to early termination of breastfeeding. Specifically, infections of the nipple or breast (including but not limited to bacterial or fungal infection of the nipple, dermatitis, eczema or bacterial or fungal mastitis) may compromise the mother's ability to effectively continue breastfeeding. Mastitis, for example, is often difficult to diagnose in its early stages, as many of its symptoms are similar to the symptoms of a common flu. As such, misdiagnosis frequently occurs in the early stages of mastitis—the stage when treatment of mastitis is most effective in allowing the mother to continuously breastfeed.

In addition, banking of donated breastmilk provides an important resource for families where the own mother's breastmilk is not available. However, much remains to be studied about how the cellular and biochemical composition of breastmilk is altered in response to infection of the mother and/or the breastfed infant. The available literature, for example, identifies great variability in the leukocyte and cytokine content of mature breastmilk between women. This variation appears to be due to the fact that most studies available so far have not appropriately considered the infection status of the mother and indeed, the breastfed infant. In other words, it is not known whether there is a normal, baseline level of leukocytes and other immune factors in breastmilk of healthy mother-infant dyads.

There is a need in the art for improved methods of assessing the infection status of a subject as well as for determining the normal, baseline cellular and biochemical composition of breastmilk in order to assess whether breastmilk has been obtained from a healthy subject.

It is an object of the present invention to overcome or ameliorate at least one of the disadvantages of the prior art, or to provide a useful alternative.

### SUMMARY OF THE INVENTION

In a first aspect the present invention relates to a method of assessing the infection status of a subject, who can be a breastfeeding mother and/or of an infant being breastfed by said mother, said method comprising the steps of:

a) analysing the composition of cellular and/or humoral immunological markers in breastmilk obtained from said breastfeeding mother; and

b) comparing said composition with a reference,

wherein a difference in the composition of immunological markers in said breastmilk when compared to said reference is indicative of a change in the infection status of said mother and/or said infant.

In certain embodiments, the reference is the composition of immunological markers in breastmilk obtained from the same breastfeeding mother obtained when the mother was healthy. Alternatively, the reference is the composition of immunological markers in breastmilk obtained from a population of breastfeeding mothers obtained when these mothers were healthy.

In one embodiment the immunological marker is selected from immune cells (IC), cytokines, and immunoglobulins.

In some embodiments analysing the composition of immunological markers comprises determining the proportion of immune cells (ICs) in the breastmilk and an increase in the proportion of said ICs when compared to the reference indicates that the mother and/or the infant have an infection.

Similarly, in some embodiments analysing the composition of immunological markers comprises determining the relative proportions of an immune cell (IC)-type within the total IC population in the breastmilk to establish an IC-type profile of the breastmilk and wherein a relative change in the IC-type profile when compared to the reference indicates that the mother and/or the infant have an infection.

In some embodiments the ICs are leukocytes and the proportion of leukocytes within the reference is between 0 and 2%. The increase in the proportion of ICs can be an increase in leukocytes of up to about 94% leukocytes of total breastmilk cells.

In some embodiments analysing the composition of immunological markers comprises determining the cytokine level in the breastmilk. Analysing the composition of immunological markers can comprise determining the relative cytokine proportions in the breastmilk to establish a cytokine profile of the breastmilk.

In yet other embodiments analysing the composition of immunological markers comprises determining the immunoglobulin level in the breastmilk. Analysing the composition of immunological markers can comprise determining the relative immunoglobulin proportions in the breastmilk to establish an immunoglobulin profile of the breastmilk.

In a second aspect the present invention relates to a method of assessing the infection status of a subject, who can be a breastfeeding mother and/or of an infant being breastfed by said mother, said method comprising the steps of:
a) determining the proportion of immune cells (ICs) in breastmilk obtained from said breastfeeding mother; and
b) comparing said proportion of ICs with a reference,
   wherein an increase in the proportion of said ICs when compared to said reference indicates that said mother and/or said infant have an infection.

In a third aspect the present invention relates to a method of assessing the infection status of a subject, who can be a breastfeeding mother and/or of an infant being breastfed by said mother, said method comprising the steps of:
a) determining the relative proportions of an IC-type within the total IC population in breastmilk obtained from said breastfeeding mother to establish a IC-type profile of said breastmilk; and
b) comparing said IC-type profile with a reference,
   wherein a relative change in the IC-type profile when compared to said reference indicates that said mother and/or said infant have an infection.

In a fourth aspect the present invention relates to a method of assessing the infection status of a subject, who can be a breastfeeding mother and/or of an infant being breastfed by said mother, said method comprising the steps of:
a) determining the cytokine level in breastmilk obtained from said breastfeeding mother; and
b) comparing said cytokine level with a reference,
   wherein a change in the cytokine level when compared to said reference indicates that said mother and/or said infant have an infection.

In a fifth aspect the present invention relates to a method of assessing the infection status of a subject, who can be a breastfeeding mother and/or of an infant being breastfed by said mother, said method comprising the steps of:
a) determining the relative cytokine proportions in breastmilk obtained from said breastfeeding mother to establish a cytokine profile of said breastmilk; and
b) comparing said cytokine profile with a reference,
   wherein a change in said cytokine profile when compared to said reference indicates that said mother and/or said infant have an infection.

In a sixth aspect the present invention relates to a method of assessing the infection status of a subject, who can be a breastfeeding mother and/or of an infant being breastfed by said mother, said method comprising the steps of:
a) determining the immunoglobulin level in breastmilk obtained from said breastfeeding mother; and
b) comparing said immunoglobulin level with a reference,
   wherein a change in the immunoglobulin level when compared to said reference indicates that said mother and/or said infant have an infection.

In a seventh aspect the present invention relates to a method of assessing the infection status of a subject, who can be a breastfeeding mother and/or of an infant being breastfed by said mother, said method comprising the steps of:
a) determining the relative immunoglobulin proportions in breastmilk obtained from said breastfeeding mother to establish an immunoglobulin profile of said breastmilk; and
b) comparing said immunoglobulin profile with a reference,
   wherein a change in said immunoglobulin profile when compared to said reference indicates that said mother and/or said infant have an infection.

In an eighth aspect the present invention relates to a method of diagnosing an infection of the breast in a lactating human subject, comprising the steps of:
a) determining the proportion of immune cells (ICs) in breastmilk obtained from said subject; and
b) comparing said proportion of ICs with a reference,
   wherein said reference is the proportion of ICs in breastmilk from the same subject obtained when said subject was healthy, and wherein an increase in the proportion of said ICs when compared to said reference is indicative of an infection of the breast of said subject.

In some embodiments, the infection is influenza. Typically, the mother suffers the influenza infection. Alternatively, both the mother and infant suffer the influenza infection whereas in other embodiments only the infant suffers the influenza infection.

In a ninth aspect the present invention relates to a method of diagnosing an infection of the breast in a lactating human subject, comprising the steps of
a) determining the proportion of immune cells (ICs) in breastmilk obtained from said subject; and
b) comparing said proportion of ICs with a reference,
   wherein said reference is the proportion of ICs in breastmilk obtained from a population of lactating human subjects when said subjects were healthy, and wherein an increase in the proportion of said ICs when compared to said reference is indicative of an infection of the breast of said subject.

In other embodiments, the infection is a bacterial or fungal infection, for example an infection localised to the breast such as mastitis. Typically, the mastitis is caused by blocked ducts.

The methods of the present invention may also detect and assess an infection that is systemic or that affects distal organs and/or tissues, for example a maternal urinary tract infection, a maternal vaginal infection, a maternal ear infection or a maternal gastrointestinal infection. In situations where the infection does not originate in the breast the number of ICs in breastmilk or the relative proportions of different type of ICs (lymphocytic and/or myeloid in origin) in breastmilk will be different to that observed when the infection originates in the breast or is localised to the breast. Hence the methods of the present invention may be used to determine the source and/or origin of the infection as well as the type of infection.

Similarly, the number of ICs in breastmilk or the relative proportions of different types of ICs in breastmilk will differ depending on the type of organism causing the infection, i.e. viral, bacterial or fungal. Hence, the methods of the present invention are able to differentiate between different types of infection.

In one embodiment, the cytokine level determined is the level of at least one of the following interleukins: Interleukin-2 (IL2); Interleukin-4 (IL4); Interleukin-6 (IL6); Interleukin-10 (IL10); and Interleukin-17A (IL17A). The cytokine level determined can be the level of at least one of the following: Interferon-gamma (IFN-γ) or Tumor Necrosis Factor (TNF).

In a further embodiment, the composition of the breastmilk is analysed by determining the relative amount of an immunoglobulin (Ig) in the breastmilk. The Ig can be IgA. The IgA can also be a secretory IgA. The Ig can also be IgG or IgM.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

In the context of this specification the following terms are defined as follows:

“comprising”—Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“infection status”—In the context of the present invention this phrase is intended to encompass the presence of a fungal, bacterial and/or viral infection, and include the location of the infection, for example an infection localised to the breast or an infection that affects a distal organ and/or tissue or is a systemic infection

“immunological marker”— In the context of the present invention this term is intended to encompass both cellular and humoral immune markers, and includes cellular immune components (immune cells or ICs) such as cells of the lymphoid and myeloid lineage (eg. B, T and NK lymphocytes, monocytes, macrophages, neutrophils, basophils, eosinophils and the like), cytokines (eg. Interleukins, interferons, TNF and the like) and immunoglobulins (eg. IgA, IgG and IgM).

“IC-type profile”— This phrase is intended to convey the concept of relative proportions of different immune cell types in breastmilk as an indicator of both infection type as well as localisation of infection.

“cytokine profile”— This phrase is intended to convey the concept of relative proportions of different types of cytokines in breastmilk as an indicator of both infection type as well as localisation of infection.

“immunoglobulin profile”—This phrase is intended to convey the concept of relative proportions of different types of immunoglobulins in breastmilk as an indicator of both infection type as well as localisation of infection.

### BRIEF DESCRIPTION OF THE DRAWINGS

Embodiments of the invention will now be described, by way of example only, with reference to the accompanying drawings in which:

FIG. 1 schematically illustrates the different cell types present in breastmilk.

FIG. 2 schematically illustrates the different processing steps employed to analyse the composition of breastmilk.

FIG. 3 shows the change in the proportion of immune cells (ICs) in response to infection; in this case the amount of ICs in breastmilk obtained during infection (mastitis) is compared to the amount of ICs in breastmilk obtained from the same woman before the infection.

FIG. 4 illustrates that the ICs identified respond to Antigen and phytohaemaglutinin (PHA) exposure in cell proliferation assays.

FIG. 5 shows, in a similar fashion to FIG. 3, the change in the proportion of immune cells (ICs) in response to infection; in this case the infection is a cold from which both the mother and the infant suffer. This figure also shows the IC profiles for breastmilk obtained from mothers before, during and after a cold which affected both the mother and the infant.

FIG. 6 and FIG. 7 show the IC profiles for breastmilk obtained from mothers before, during and after a cold which affected both the mother and the infant.

FIG. 8 shows the effect of an infant-only cold on the IC composition of the mother.

FIG. 9 illustrates the IC profiles of breastmilk from three mothers during late stages of lactation showing that no or only few ICs are detectable during late lactation in healthy subjects.

FIG. 10 compares the IC composition of a mother's colostrum with the IC composition of breastmilk obtained during week 2 of lactation.

FIG. 11 shows a further example of the comparison of the IC composition of a mother's colostrum with the IC composition of breastmilk obtained at a later stage illustrating the decline of ICs during the maturation of breastmilk.

FIG. 12 shows a further example similar to the example illustrated in FIG. 11.

FIG. 13 shows the proportion of ICs (in %) in relation to total cell numbers in breastmilk obtained from infected and healthy mothers as well as the proportion of ICs in colostrum obtained from a healthy mother.

FIG. 14 illustrates the IC composition of breastmilk obtained from infected as well as healthy subjects during early and late stages of lactation (healthy=bottom; infection=top).

FIG. 15 illustrates the IC composition of breastmilk by infection status.

FIG. 16 illustrates the proportion of viable immune cells obtained from infected as well as healthy subjects during early and late stages of lactation (healthy=red/bottom; infection=blue/top).

FIG. 17 illustrates total cell count in breastmilk by infection status.

FIG. 18 illustrates total cell count in breastmilk obtained from infected as well as healthy subjects during early and late stages of lactation (healthy=red/the curve moving upward around week 100; infection=blue/the curve moving downward around week 100).

FIG. 19 shows the total cell counts in breastmilk obtained from each participant when infected as well as when healthy.

FIG. 20 illustrates the amount of IgA detected in breastmilk from healthy as well as from infected subjects and in colostrum.

FIG. 21 shows the amounts of IgA detected in breastmilk obtained from infected as well as healthy subjects during early and late stages of lactation (healthy=red/bottom; infection=blue/top).

FIG. 22 illustrates the amount of IgG detected in breastmilk from healthy as well as from infected subjects and in colostrum.

FIG. 23 illustrates the amount of IgG detected in breastmilk from healthy as well as from infected subjects and in colostrum (healthy=red/the bottom curve at week 50; infection=blue/the top curve at week 50).

FIG. 24 illustrates the amount of IgM detected in breastmilk from healthy as well as from infected subjects and in colostrum.

FIG. 25 illustrates the amount of IgM detected in breastmilk from healthy as well as from infected subjects and in colostrum.

FIG. 26 shows the amount of IgM detected in breastmilk obtained from each participant when infected as well as when healthy.

FIG. 27 shows the Lactoferrin concentrations in breastmilk from healthy as well as from infected subjects and in colostrum.

FIG. 28 shows the Lactoferrin concentrations in breastmilk obtained from infected as well as healthy subjects during early and late stages of lactation (healthy=red/bottom; infection=blue/top).

FIG. 29 shows the Lactoferrin concentrations in breastmilk obtained from each participant when infected as well as when healthy.

FIG. 30 shows the cytokine levels in breastmilk in response to infection.

FIG. 31 illustrates different IC populations present in breastmilk in response to infection.

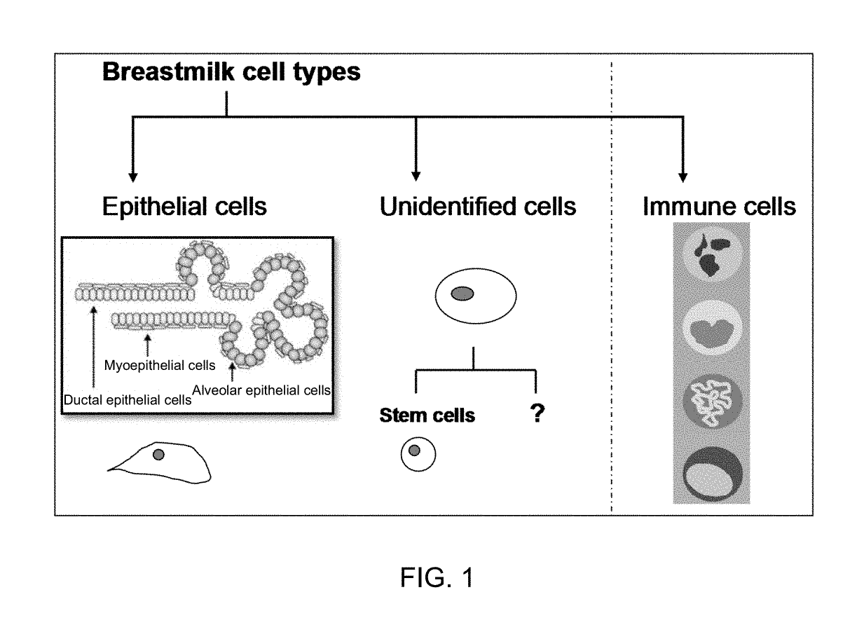

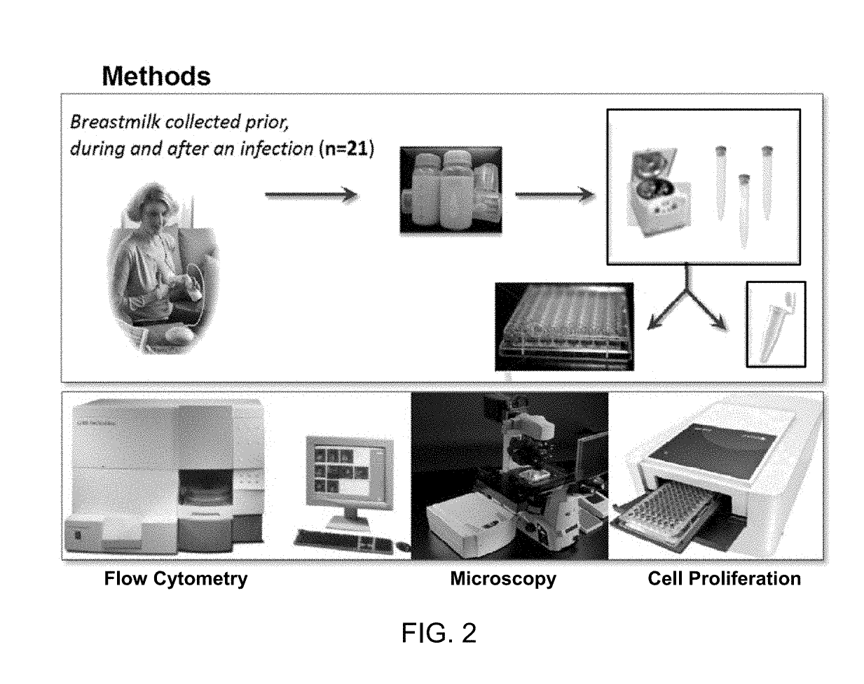

### DETAILED DESCRIPTION OF THE INVENTION

The prior art indicates that colostrum has a high content of immune cells even when obtained from a healthy lactating subject. Further, it has been shown that colostrum contains high levels of secretory immunoglobulin A (IgA) and lactoferrin.

However, it has unexpectedly been found that the composition of mature breastmilk, especially with respect to the proportion of immune cells and immune factors, is not as variable as long thought. Rather than having a high variability, it has been found that mature human breastmilk contains no or very few immune cells under normal conditions. Specifically, it has been found that typically less than 2% of all cells contained in breastmilk from a healthy lactating subject are immune cells.

Further, it has been established that a change in infection status triggered by, for example a maternal infection, leads to a rapid increase in the immune cell content of mature breastmilk, which, in turn, returns close to baseline levels upon recovery. However, a residual elevation above baseline levels may be indicative of a previous infection.

In addition, it has surprisingly been found that the immune cell content of mature breastmilk remains at baseline levels throughout the entire lactation period in healthy individuals.

Similarly, it has been found that cytokine levels as well as the levels of other immune factors such as immunoglobulins change in response to infection.

The baseline ranges of immune cell levels and other immune factor levels in breastmilk have been examined in healthy mother/infant dyads. It has also been examined how these levels are influenced by infections of the mother/infant dyad.

As indicated above, the present invention relates to the surprising finding that the composition of mature breastmilk, especially with respect to the proportion of immunological markers such as immune cells, cytokines and immunoglobulins, is not as variable as long thought. Rather than having a high variability, it has been found that mature human breastmilk contains no or very few immune cells under normal conditions. Specifically, it has been found that typically less than 2% of all cells contained in breastmilk from a healthy lactating subject are immune cells.

Similarly, it has been found that cytokine levels as well as the levels of other immune factors such as immunoglobulins increase in response to infection.

While it has been confirmed that colostrum has a high content of immune cells even when obtained from a healthy lactating subject, it has also been established that infection triggers a rapid increase in the immune cell content of mature breastmilk. However, this rapid increase returns to near pre-infection baseline levels upon recovery. Specifically, it has been found that colostrum contains considerable numbers of immune cells (leukocytes—13-70% out of total cells) and high levels of immunoglobulins and lactoferrin. Within the first 1-2 weeks postpartum, leukocyte numbers decreased significantly to the above-mentioned low baseline level in mature breastmilk of 0-2% (P<0.001).

Further, it has been surprisingly found that the immune cell content of mature breastmilk remains at near baseline levels throughout the entire lactation period in healthy individuals. Specifically, this baseline level was maintained throughout lactation unless the mother and/or her infant became infected. In the case of infection leukocyte levels significantly increased up to 94% out of total cells (P<0.001). Upon recovery from the infection, baseline values were restored.

As indicated above, the methods of the present invention allow for the assessment of a subject's infection status based on the immune cell (IC) composition of the subject's breastmilk. This will allow early diagnosis of infections of the breast but also of an infection that is systemic or that affects distal organs and/or tissues remote from the breast. Especially mastitis, either a fungal of bacterial infection, is a serious complication during breastfeeding and, if advanced, is severely debilitating condition. The early symptoms of mastitis, however, are very similar to the symptoms of the common flu and, as a result, mastitis is often only appropriately diagnosed once advanced. This often compromises continuous breastfeeding and can in instances lead to early termination of breastfeeding.

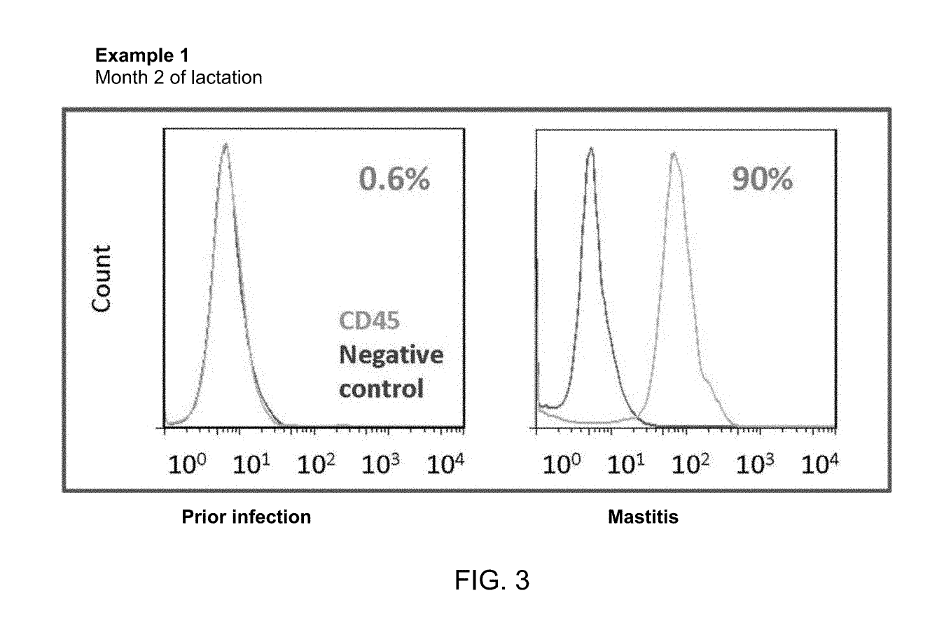

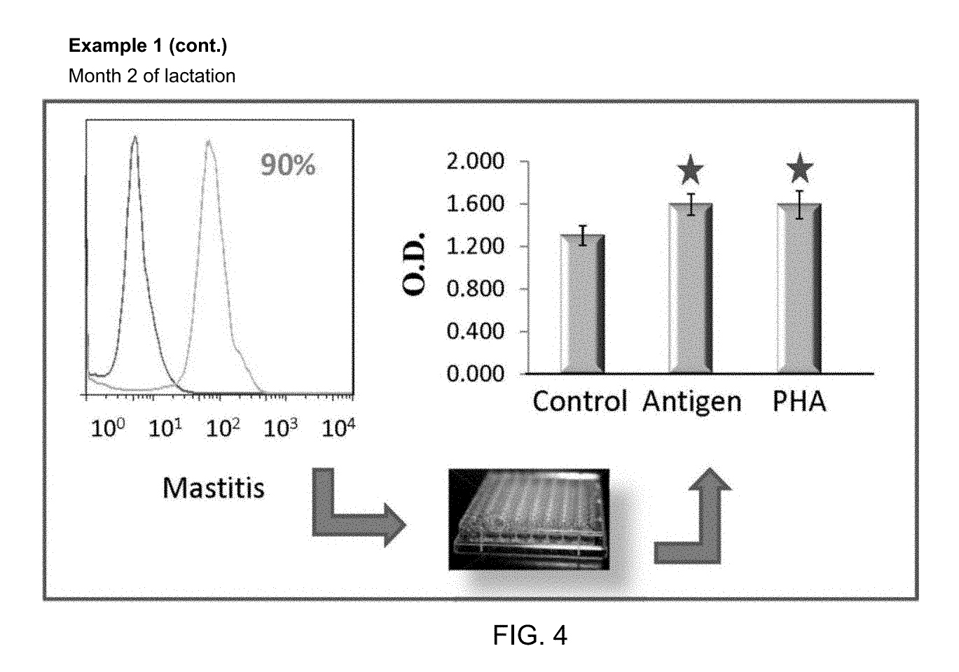

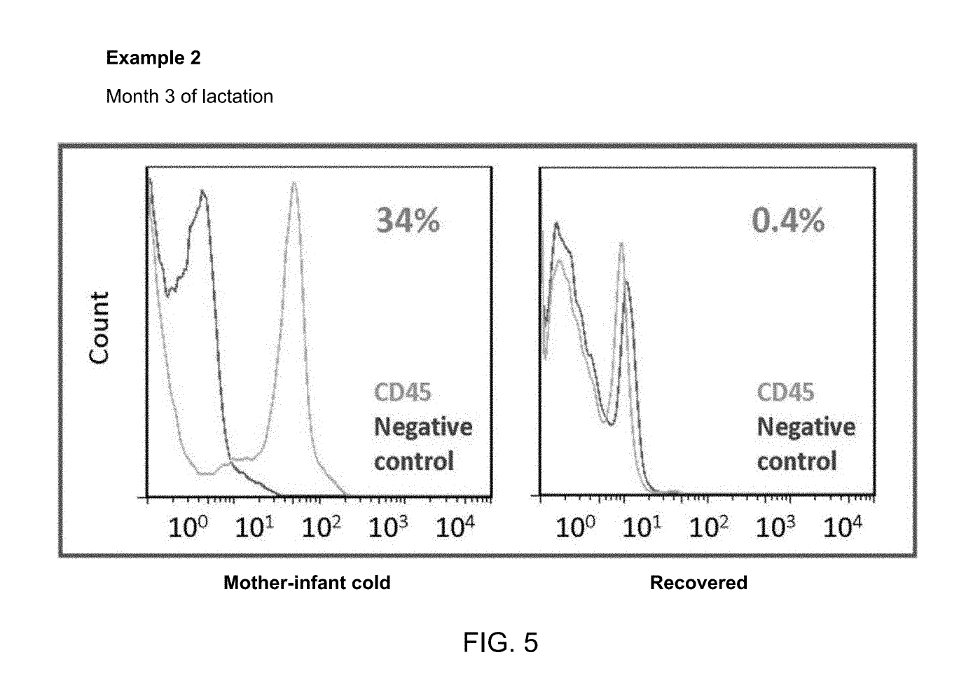

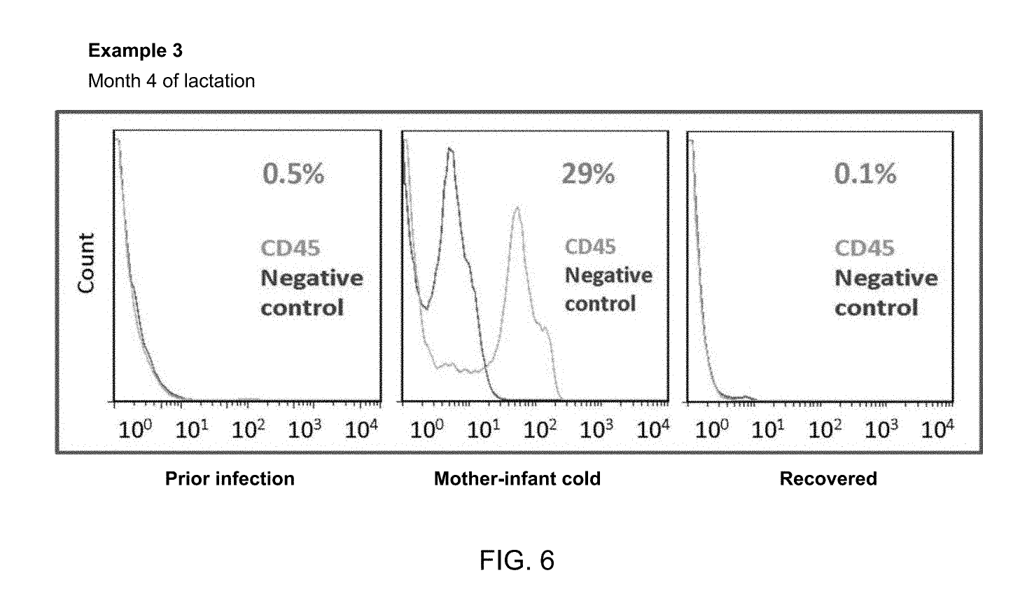

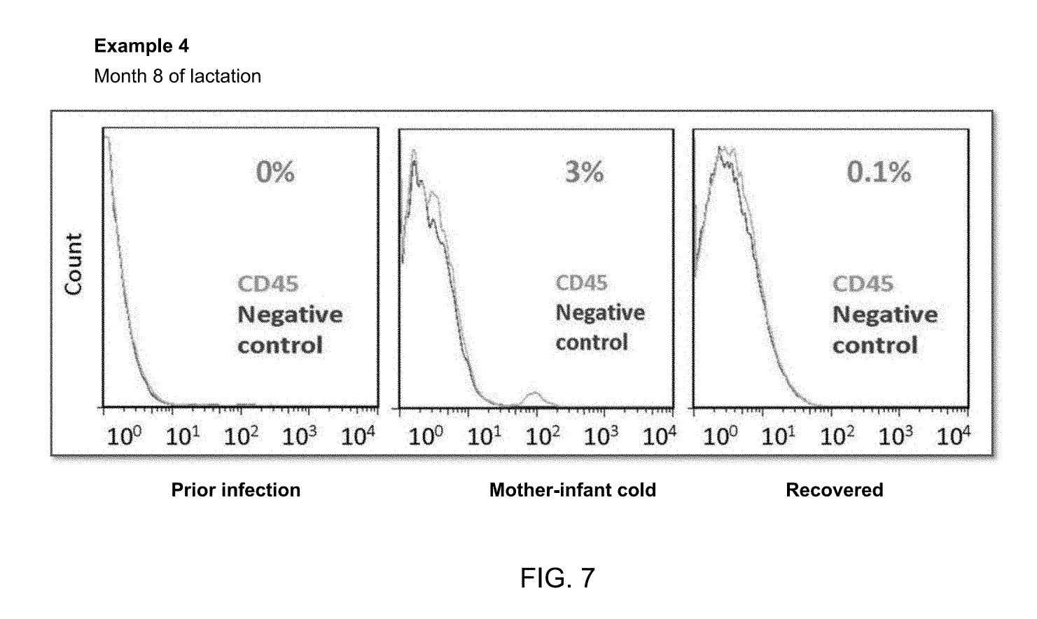

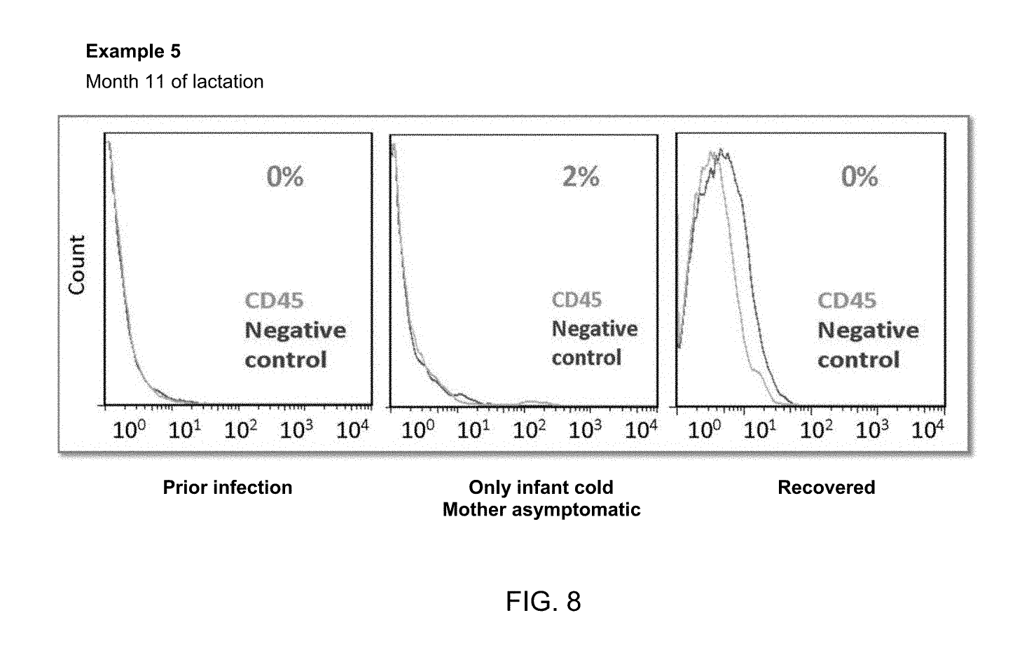

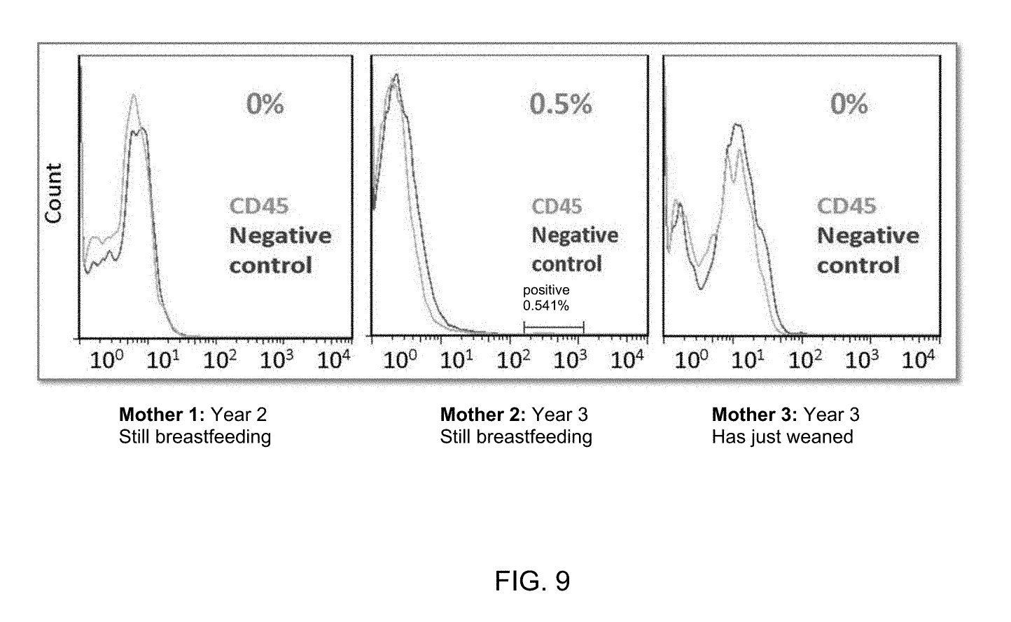

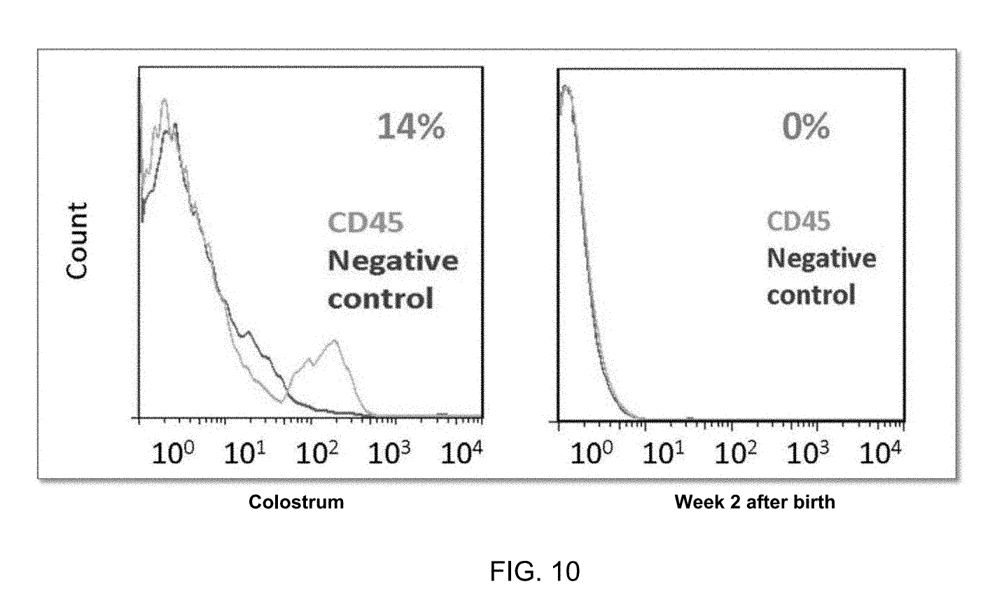

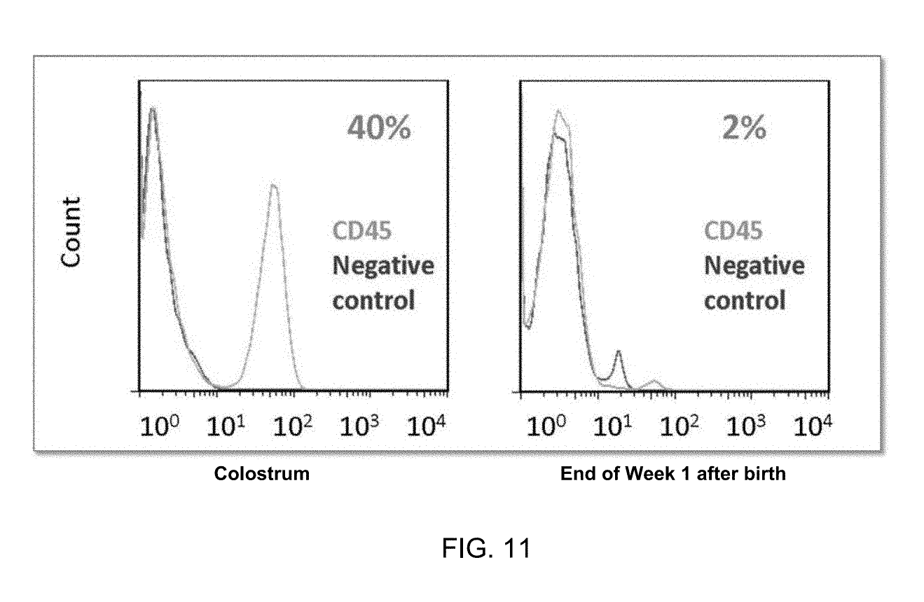

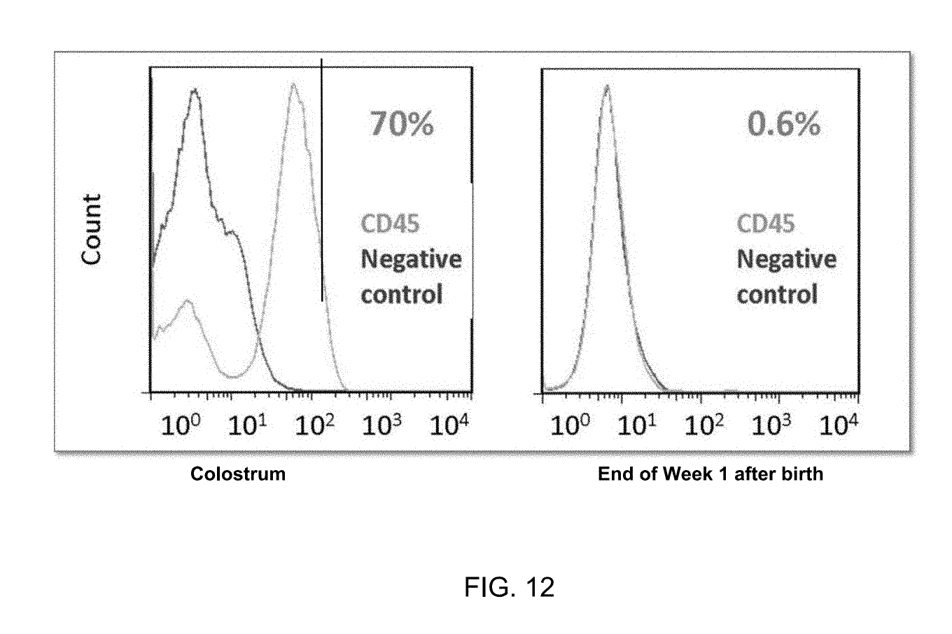

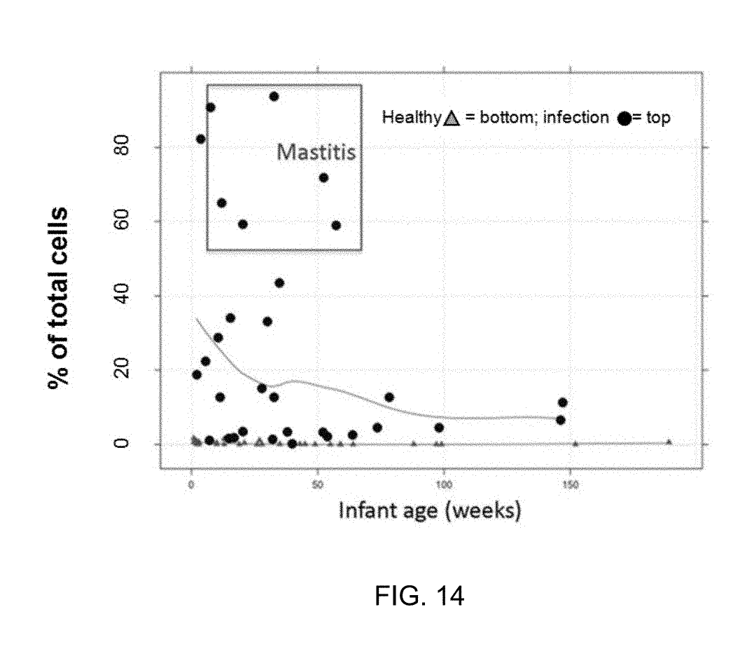

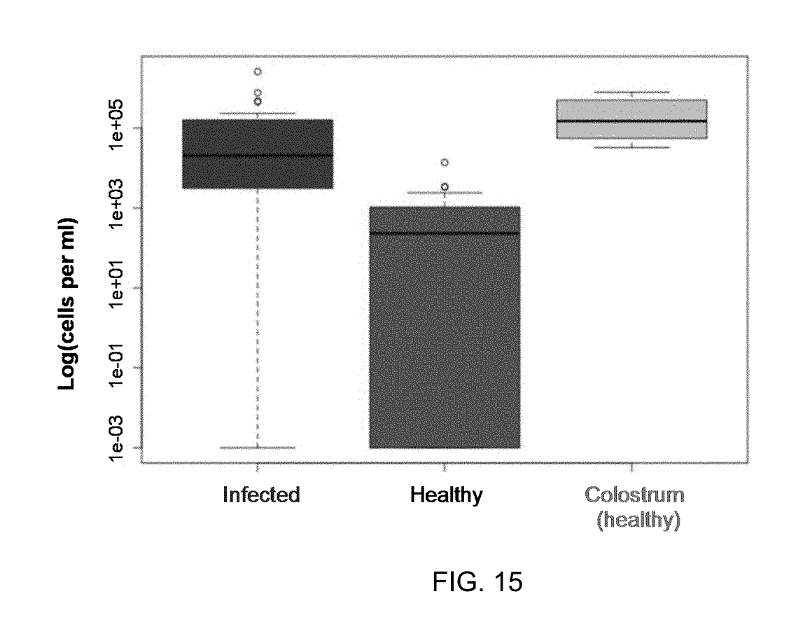

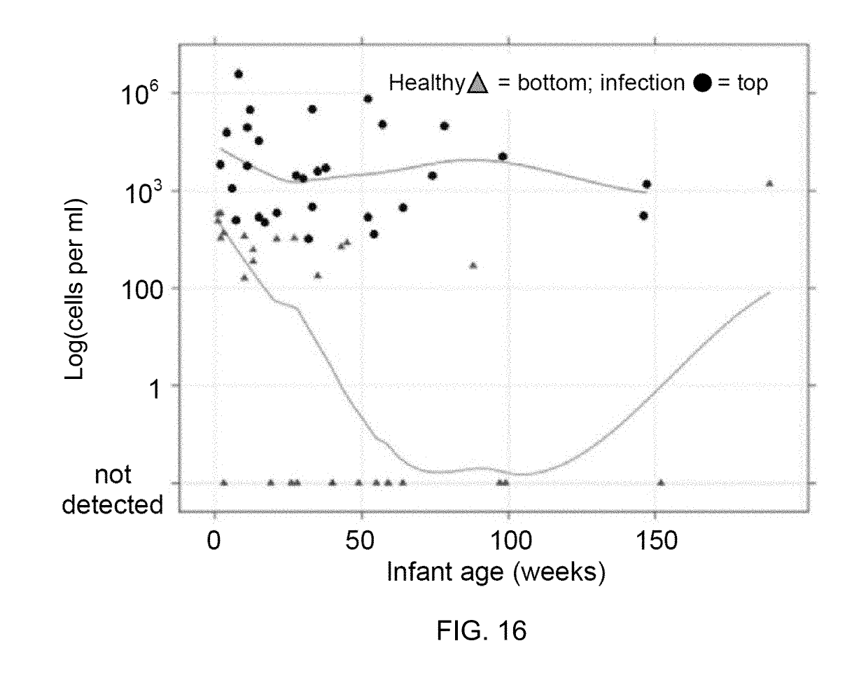

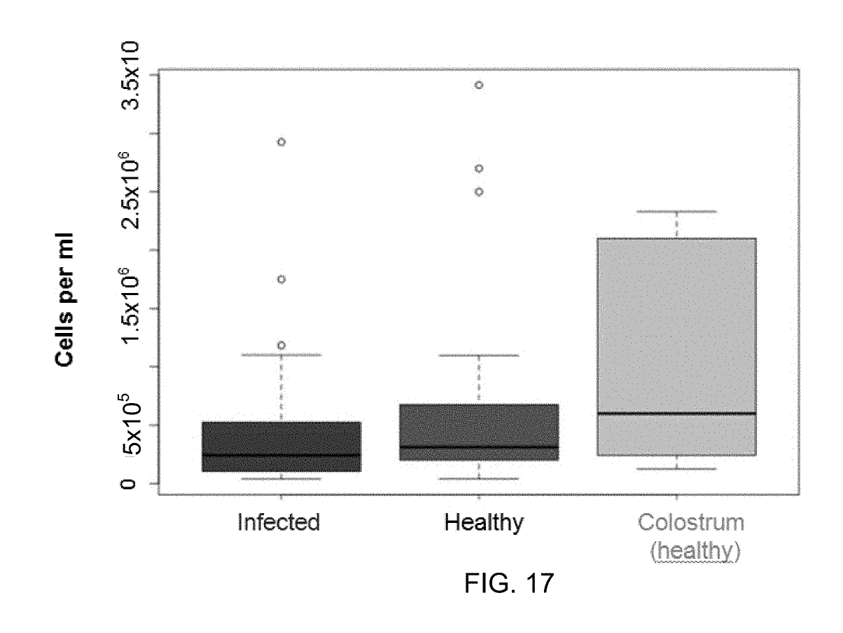

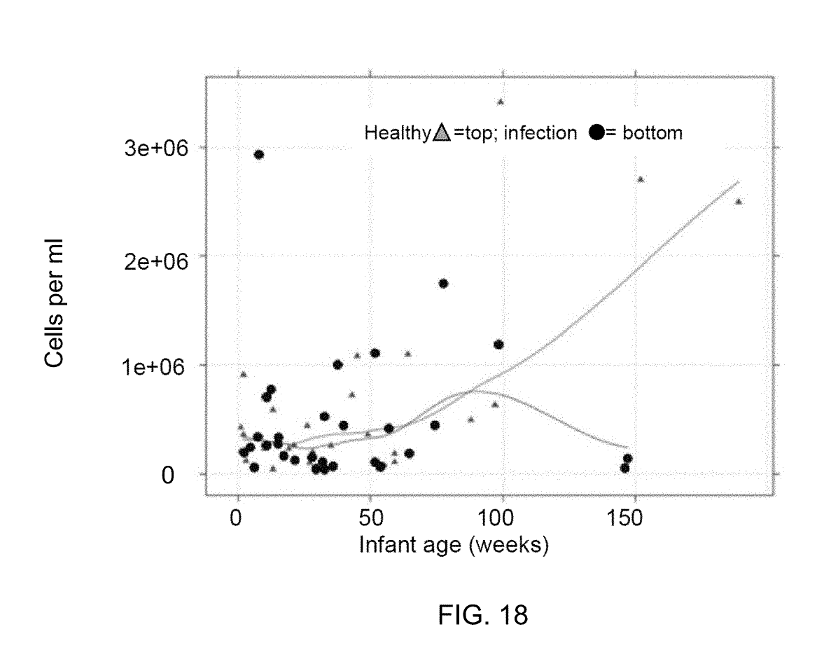

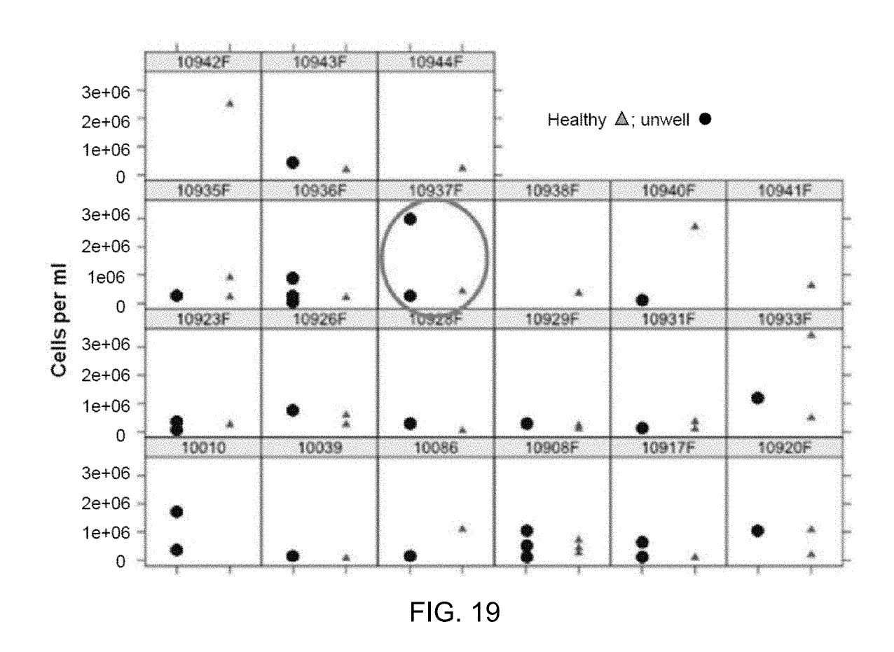

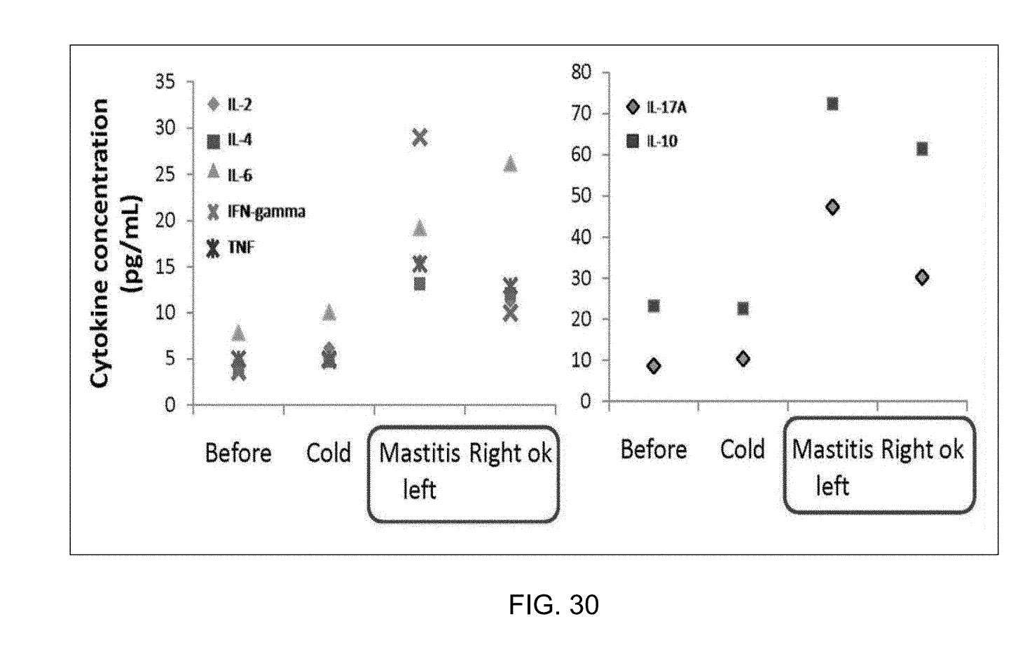

Referring to FIGS. 3, 4, 14 and Table 1 below, it is noteworthy that the IC content of mature breastmilk increases drastically in response to a mastitis. Interestingly, whereas the increase and overall amount of ICs in mature breastmilk is less pronounced in response to maternal cold (also see FIGS. 5 to 7). Again some cytokines are drastically increased in response to mastitis. Specifically, and as for example shown in FIG. 30, the levels of Interleukin-2 (IL2); Interleukin-4 (IL4); Interleukin-6 (IL6); Interleukin-10 (IL10); and Interleukin-17A (IL17A) as well as of Interferon-gamma (IFN-γ) and Tumor Necrosis Factor (TNF) increase in response to mastitis. While some of these cytokines are also increased in response to a maternal cold, the difference in cytokine levels when compared to the reference, baseline levels are again greater in response to mastitis.

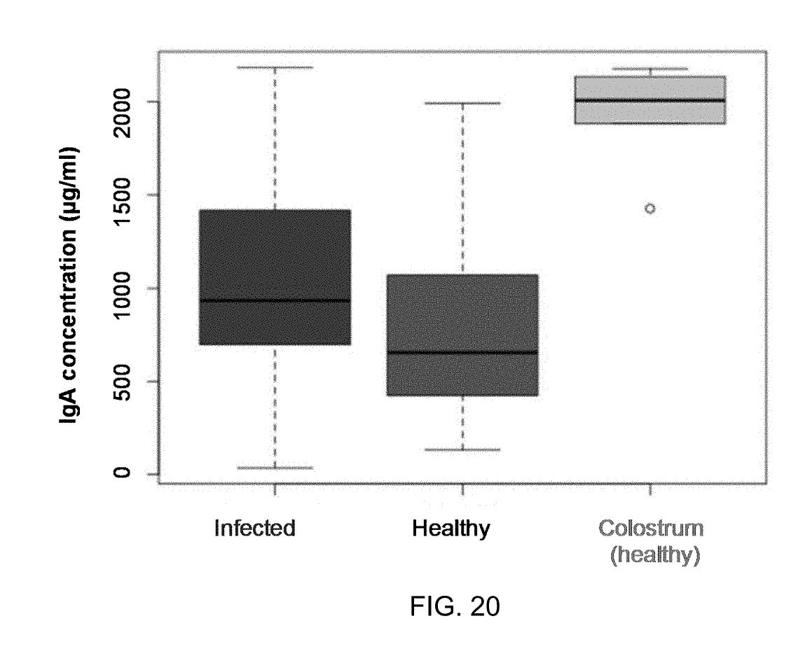

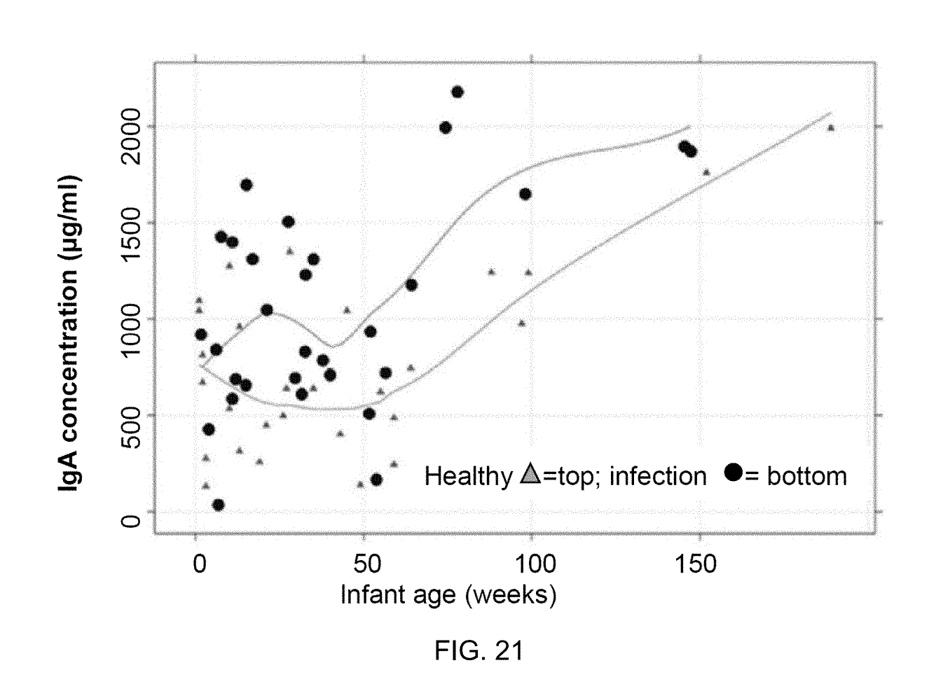

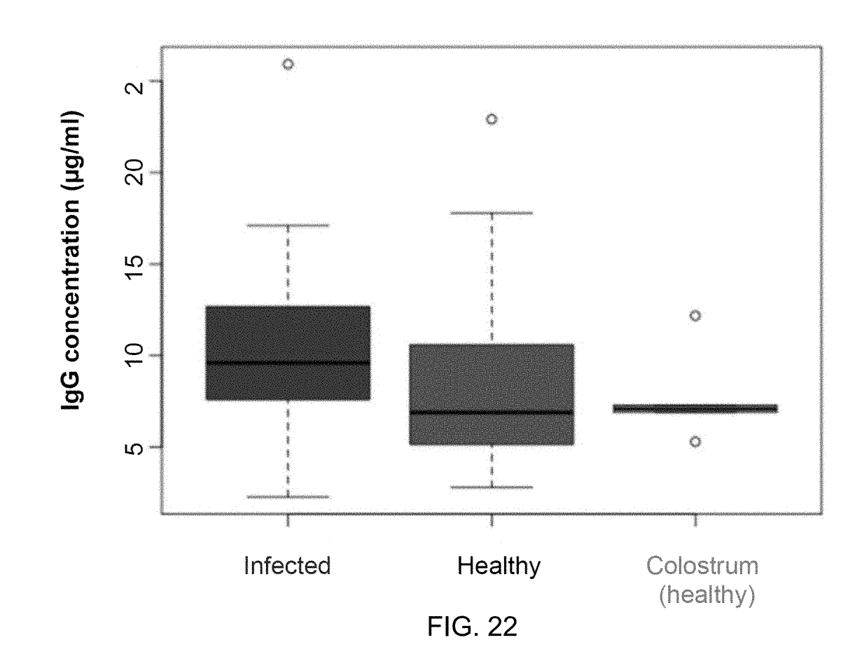

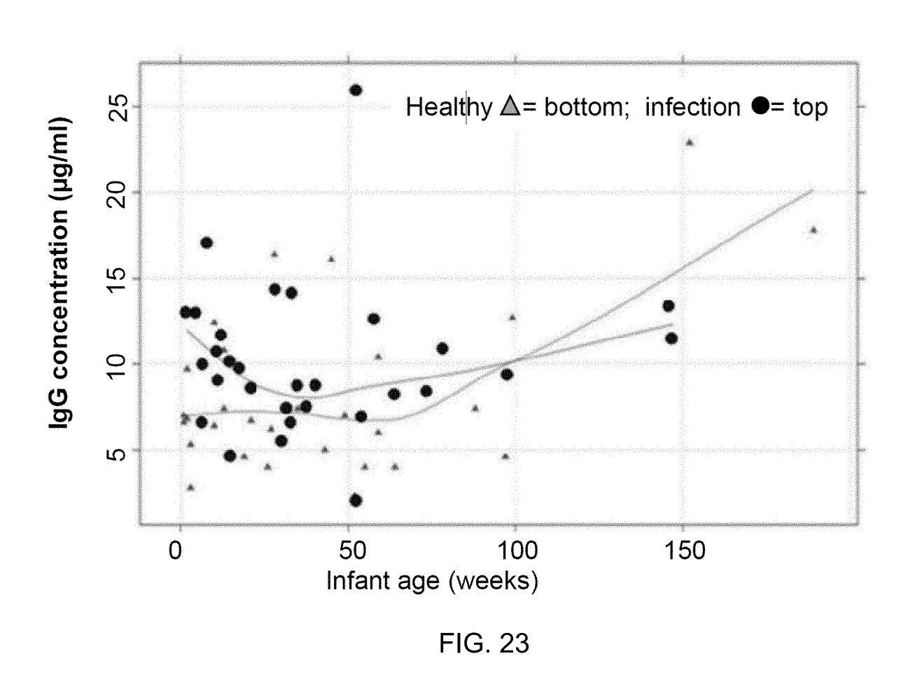

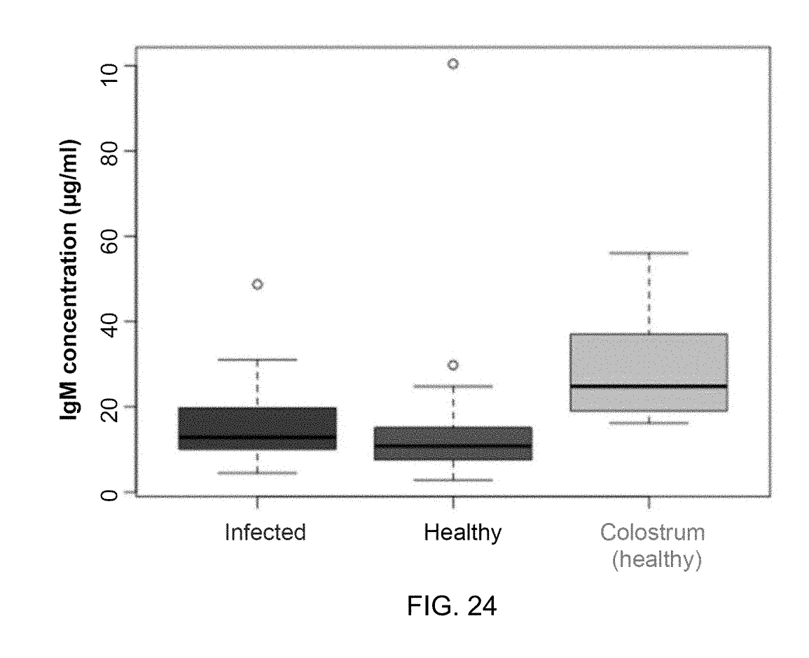

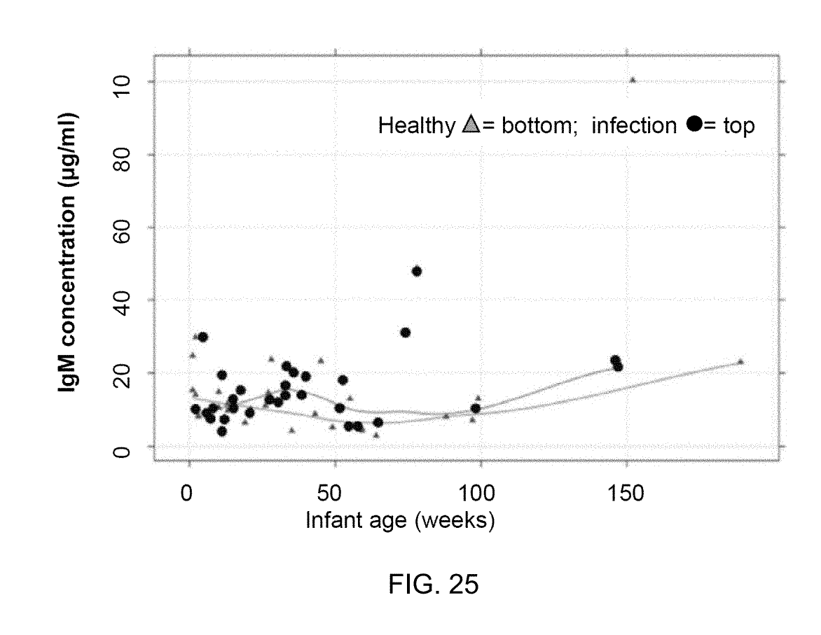

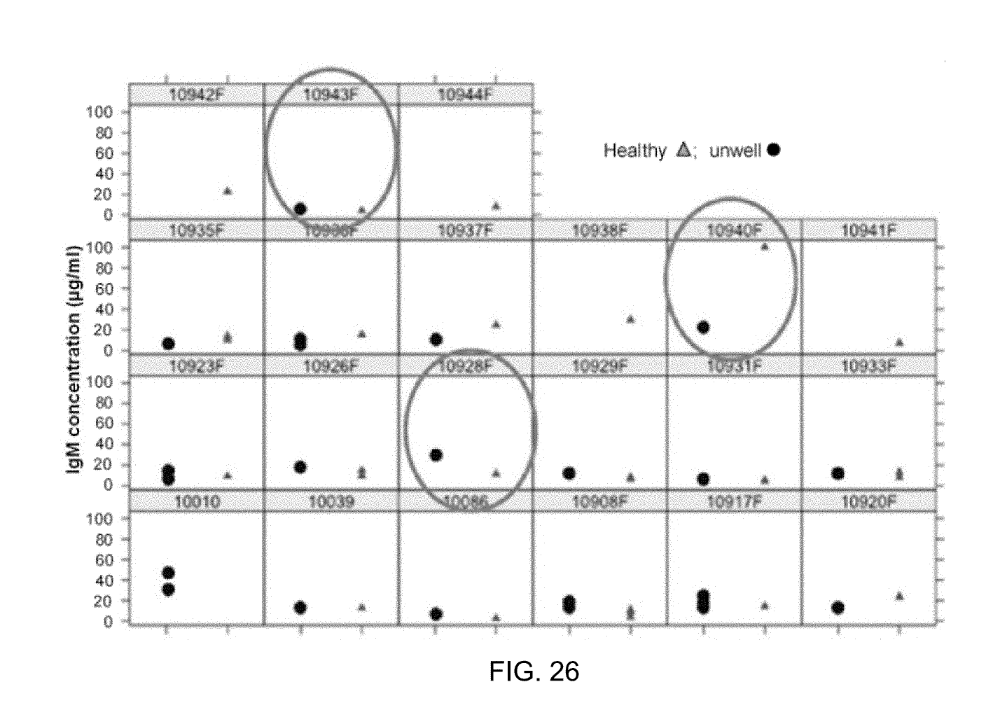

Similarly, as can be seen in FIGS. 20 to 26, the concentrations of immunoglobulins such as for example IgA, IgG and IgM in breastmilk also change in response to infection.

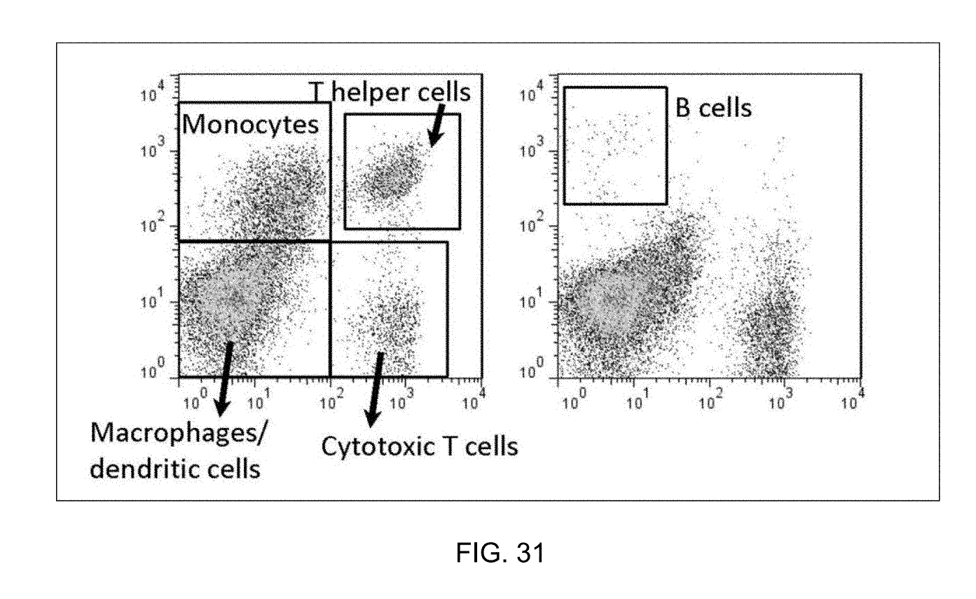

As such, analysing the composition of breastmilk not only allows a general assessment of a change in the infection status, but it also allows for the differentiation between different infection types due to characteristic IC, cytokine and immunoglobulin amounts, as well as characteristic distribution of IC-types within the total IC population (see FIG. 31), cytokine or immunoglobulin profiles.

In practice the methods of the present invention cannot only assist in the diagnosis but can also serve as a further tool to assess the effectiveness of a treatment. For example, if a lactating mother is receiving treatment, relatively simple analysis of her breastmilk in accordance with the present invention will reveal whether the treatment is effective, i.e. whether a decline in ICs in the breastmilk and/or an appropriate change in the cytokine and/or immunoglobulin profiles can be observed. This is particularly important for infections, which can be caused by either bacterial of fungal pathogens. Fungal mastitis for example does not respond to antibiotic treatment which can easily be assessed by a method of the present invention.

Furthermore, an increasing number of bacterial strains display resistance to commonly used antibiotics. An early indication as to whether a chosen antibiotic is in fact effective to treat the particular bacterial pathogen can be obtained by analysing the composition of breastmilk in accordance with the present invention. Such analysis will allow the treating physician to change the antibiotic at an earlier stage than currently possible and without the need for the patient to go through an entire treatment regime with an ineffective antibiotic before switching to a more effective antibiotic.

Also as indicated above, the methods of the present invention can be utilised to assess a change in the infection status of a lactating subject in response to infection. Without limitation, a change in infection status in response to infections such as influenza, fungal or bacterial mastitis, urinary tract infection, vaginal infection, ear infection or gastrointestinal infection can be assessed by the methods of the present invention.

In addition to the above, it is common that breastmilk donated from lactating mothers is banked as a resource for families where breastmilk from the own mother is not available. However, even with the careful assessment of the donor and her infection status latent and/or asymptomatic infections may not become apparent. However, as has been shown by the present studies, even latent and/or asymptomatic infections can severely alter the IC composition and/or the cytokine and/or immunoglobulin profiles of breastmilk.

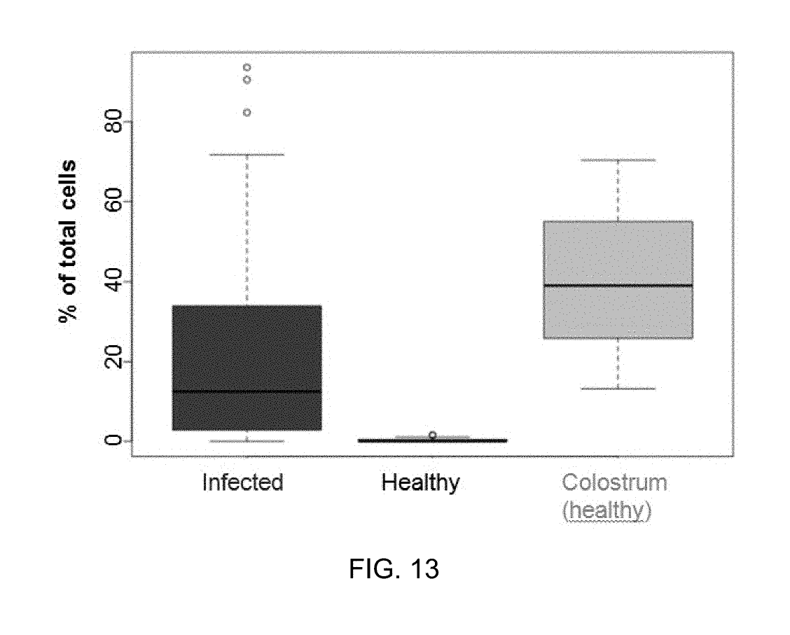

In particular, it has been shown (FIGS. 13, 20, 22, 24 and 30) that the amount of ICs, as well as of cytokines and/or immunoglobulins increases in response to infection. However, as the recipients of the donated and banked breastmilk are generally not closely related to the donor, the immunological load of breastmilk obtained from an infected donor may lead to an undesirable immune response by the recipient. The methods of the present invention provide for convenient screening of breastmilk donations for their suitability for banking and future heterologous consumption.

### Materials & Methods

### Breastmilk Sample Collection

A longitudinal study was designed and approved by the Human Research Ethics Committee of The University of Western Australia. Written informed consent was obtained from all participants who included volunteering breastfeeding mothers of a broad lactation spectrum of 1 to 21 months, recruited prior or during an infection. The study included collection of a breastmilk sample of 10-50 ml principally on 2-3 different time points per participant: prior, during and after an infection, with the first time point missing in five of the participants. Three of the participants underwent infection incidents on two occasions during the study period, for both of which breastmilk samples were acquired. In these cases, the sampling sequence was: prior, during and after infection 1, during and after infection 2. One of the participants suffered repeated infections during the study period (cold, mastitis, urinary tract infection and cold) during all of which breastmilk samples were acquired. Finally, one of the participants never experienced an infection during the study period and in this case one control sample was obtained from this individual.

The mothers provided detailed description of the symptoms of the infections, which were general or respiratory viral or bacterial infections of the mother and/or the breastfed infant, such as influenza (sore throat, fever, blocked/runny nose, coughing, sneezing), mastitis, nipple thrush, gastrointestinal tract infection (nausea, diarrhoea, vomiting, headache), and urinary tract infection (frequent feeling and/or need to urinate, dysuria, hematuria, malaise).

Breastmilk samples were aseptically collected in the morning within 30 min of a feed using a Medela Symphony or Swing electric breast pump (Medela AG, Switzerland) and kept in sterile polypropylene vials at approximately 20° C. during transportation to the laboratory. Cellular analyses were conducted in the freshly expressed samples within 1-2 hours of breastmilk expression, while all other analyses were done in frozen (−80° C.) sample aliquots within five months of sample collection as described below.

### Subjects

All participants completed the study's questionnaire. Socioeconomic stratum, maternal age range, mean maternal age, ethnic background, employment before delivery, infant delivery, mean and range of infant birth weight, mean and range of gestational age, number of male and female infants, Apgar scores, first- or second-born.

### Cellular Analyses

Freshly expressed breastmilk samples were transported to the laboratory at 20° C. and aliquoted appropriately within 1-2 hours of expression. A 1-5 ml aliquot was stored at −80° C. for biochemical analyses, while the bulk of the sample was diluted with equal volume of sterile phosphate-buffered saline (PBS, pH 7.4, Gibco, USA) and centrifuged at 2,000 rpm 20 min at 20° C. The fat layer was removed with a pipette and the liquid part was stored separately at −80° C. for biochemical analyses. The cell pellet was washed three times in PBS and was finally resuspended in 7% Fetal Bovine Serum (FBS, Certified, Invitrogen, USA) in PBS.

First, the total cellular content and cell viability of each sample was determined with a Neubauer hemocytometer by Trypan Blue exclusion. For FACS analyses, the cell suspension was distributed in vials and the cells were incubated for 1 hour at 4° C. with mouse anti-human CD45 conjugated to fluorescein isothiocyanate (FITC) (1:11, Miltenyi Biotec, Germany; 1:50, Becton Dickinson, San Diego, USA) or phycoerythrin (PE) (1:11, Miltenyi Biotec, Germany). Negative control samples were incubated with the respective isotype control conjugate obtained from the same suppliers. Subsequently, the cells were washed twice in 7% FBS in PBS and incubated for 30 min at 4° C. with live/dead fixable far-red dead cell stain (Invitrogen, USA) as per manufacturer's instructions. This step enabled differentiation between dead and live cells, of which only the latter were taken into account in the analyses. After incubation, the cells were washed twice in 7% FBS in PBS, resuspended in 1% paraformaldehyde/0.7% sucrose solution in PBS and were either immediately analysed or stored at 4° C. for up to 12 hours prior to analysis. FACS analysis was done with a FACS Calibur Flow Cytometer (Becton Dickinson, N.J., USA) and further data analyses were done using the FlowJo software. A completely unstained sample was always prepared in which propidium iodide (Sigma-Aldrich) was added at 1 μg/ml after fixation and immediately prior to FACS analysis to check for the cellular uniformity of each cell suspension. In cases where this sample contained non-cell particles (such as cellular fragments or fat globules), propidium iodide was added in all other samples (stained and controls) of the same cell suspension and only the cellular component was taken into account in the analyses.

Part of the cell suspension obtained after centrifugation was immediately fixed in 1% paraformaldehyde/0.7% sucrose solution in PBS for immunofluorescence staining. Cytospins of the cell suspensions on glass slides were generated by centrifuging at 600 rpm for 4 min. The same antibodies used for flow cytometry were also used for immunofluorescence staining and were applied on the cytospins for 1 hour at room temperature under humid conditions. Appropriate controls were also used. The cytospins were then washed in PBS, incubated with DAPI (1:100, Dako) for 15 min, and after a final wash they were mounted in fluorescence mounting medium (Dako) and observed using an Olympus TH4-200 inverted microscope.

### Biochemical Analyses

For all biochemical analyses, defatted acellular breastmilk sample aliquots stored at −80° C. were used. Cytokines were determined using a multiplex kit (Millipore) for 20 different cytokines, chemokines and growth factors, following the manufacturer's instructions. In short, the stored breastmilk samples were thawed to room temperature and 50 μl of sample per well was added in a 96-well filter-bottomed plate. Each sample was assayed in duplicate. After addition of the beads and incubation on a plate shaker overnight at 4° C., the wells were vacuum-washed with buffer and antibody conjugate was added for a 1-hour incubation on a plate shaker. An additional incubation with streptavidin-phycoerythrin was done on the plate shaker, which was followed by a vacuum-wash and addition of 100 μl of sheath fluid. The plate was read in a Luminex 200 Multiplex Analyser (Invitrogen). A five-parameter logistic model was used to convert the bead fluorescence intensities to analyte concentrations.

Secretory IgA (sIgA), IgG and IgM were analysed by enzyme-linked sandwich immunoassay. The 96-well filter-bottomed plates were coated with 250 μl/well of rabbit anti-human sIgA (1:2000 in PBS, Gibco, USA) or mouse anti-human IgG (1:2000 in PBS, Gibco, USA) or mouse anti-human IgM (1:2000 in PBS, Gibco, USA) overnight at 4° C. Subsequently, the antibody solution was carefully discarded and 300 μl of blocking solution (5% w/v carnation soy powder in 0.05% Tween-20 in PBS) was added to each well for a 30-min incubation at room temperature. The stored breastmilk samples were thawed to room temperature and diluted in 0.05% Tween-20 in PBS 2000 times. The microtitre plate was then washed three times with washing buffer (0.05% Tween-20 in PBS) and 200 μl/well of the standards, samples or controls were added to the plate, which was then covered with a plate sealer for a 60-minute incubation at 37° C. The plate was then washed three times with washing buffer before addition of 200 μl/well of secondary antibody and incubation for 30 min at room temperature (1:1000 rabbit anti-human sIgA conjugated to horseradish peroxidase; or 1:1000 mouse anti-human IgG conjugated to biotin; or 1:1000 mouse anti-human IgM conjugated to biotin). All secondary antibodies were diluted in 0.05% Tween-20 in PBS containing 1 mg/ml BSA to prevent non-specific absorption. In the IgG and IgM assays, incubation with the secondary antibody was followed by three washes and subsequent incubation with 200 μl/well of an anti-biotin-horseradish peroxidise antibody (1:1000) for 30 min at room temperature. After incubation with the secondary antibody in the sIgA assay and with the tertiary antibody in the IgG and IgM assays, the plate was washed six times with washing buffer and then rinsed with deionised water before addition of 200 μl/well of the colour reagent (6% hydrogen peroxide, 1% ABTS). The colour reaction was stopped after 30 min by addition of 100 μl/well of 3M sulfuric acid. The plated was read at 405 nm using a plate reader.

### Statistical Analyses

Statistical analyses were performed in R 2.9.0 1 (R Foundation for Statistical Computing, Vienna, Austria, 2009) using the base packages and libraries lattice (Sarkar D., In. R package version 0.17-22 ed, 2009), nlme Pinheiro J. et al. In. R package version 3.1-89 ed, 2008) and multcomp (Hothorn T. et al. 2008). Results are presented as mean±SEM unless otherwise specified, and P<0.05 was considered significant. Linear mixed effects models were used to determine which of the response variables [leukocyte levels (% of total cells), leukocyte levels (per mL of breastmilk), total and viable cell levels (per mL breastmilk), cell viability (% viable cells of total cells), sIgA, IgG, IgM and lactoferrin] were affected by breastmilk type, lactation stage, infection status of the mother/infant dyad, and breastfeeding status (exclusive versus non-exclusive). All models included a grouping factor of dyad and a random effect of different baseline levels for each dyad. Where infection status was included in a model, ‘healthy’ was considered to be the reference level. To determine whether data transformation was required, comparisons were made between raw data, square root-transformed data and natural log-transformed data models. After fitting the univariate model for each predictor, diagnostic plots (fitted values versus standardised residuals; qq-plot of residuals) were examined. In cases where a transformation was obviously superior, this was used for all covariate models. Due to the measured zeroes obtained for leukocyte levels (% and per mL breastmilk) of some samples, these variables were transformed using the additive constant 0.5 for both the square root and the log transformations (Yamamura K. 1999). Differences between colostrum, ‘healthy’ and ‘infection’ samples were tested for with a three-level categorical predictor of breastmilk type. Differences between ‘healthy’ samples and each of the infection types were tested using a five-level factor (‘healthy’, ‘breast’, ‘baby’, ‘cold’, and ‘other’), with the colostrum samples omitted. Differences between pre- and post-infection ‘healthy’ samples were tested where data was available (n=7) by classifying ‘healthy’ as a two level factor (‘pre’ and ‘post). To determine whether there were interactions between the infection status of the dyad and either lactation stage (age in weeks) or exclusive breastfeeding (yes/no) in mature breastmilk, interaction models were considered for each of these covariates separately. Infection status was considered as a two level factor (‘heathy’ and ‘infection’). Models were selected using backwards stepwise methods, starting with the main effects+interaction model. Where the interaction was significant, all main effects were retained in the model.

For the lactation stage model, analysis was restricted to records where the baby was under 60 weeks of age (N=46 records, for 15 participants. The effect of viral antigens and PHA on proliferation rate in leukocyte cultures generated from two samples of mastitic breastmilk was also assessed using linear mixed effects models. Separate models were used for each response variable, with replicate as the grouping variable and a random effect of different baseline effects per replicate. Tukey's HSD (honestly significant difference) test was used to test all pairwise comparisons between the three treatments (control, viral antigen, PHA). Multivariate patterns in the immune markers (sIgA, IgG, IgM, lactoferrin) and three cellular measures (total cell, leukocyte number and percentage of leukocytes) were investigated analytically using singular value decomposition principal components analysis on scaled and centred data and graphically using biplots. This enabled comparisons between the conditions that were seen infrequently.

### Results

Healthy Mother/Infant Dyads have a Low Baseline Level of Leukocytes in Breastmilk

FACS quantification of CD45 protein expression was done by an analytic gating strategy that excluded interference by dead/dying cells and/or fat globules. Although colostrum always contained considerable levels of leukocytes (13.2-70.4% leukocytes of total breastmilk cells; 32,175-784,080 viable leukocytes/mL colostrum), these rapidly decreased towards the end of the first week postpartum to reach a baseline level in transitional breastmilk (0-1.7% leukocytes of total breastmilk cells; 0-3,450 viable leukocytes/mL breastmilk) and mature breastmilk (0-1.5% leukocytes of total breastmilk cells; 0-1,151 viable leukocytes/mL breastmilk) when both the breastfeeding mother and infant were healthy (Tables 2 and 3). The low baseline level of mature breastmilk leukocytes under healthy conditions was maintained even at late lactation stages (year 2-4 postpartum) (Table 2) and was found to fluctuate within certain limits (0-2% leukocytes of total breastmilk cells) both between and within individuals.

### Infections of the Mother/Infant Dyad Stimulate a Leukocyte Response in Breastmilk

Infections of the breast, other organs or general maternal infections all stimulated a leukocyte response in breastmilk that ranged from 0.7% (sore breast) to 93.6% (mastitis) leukocytes of total breastmilk cells (P<0.001) (Tables 2 and 3). Severe breast infections such as mastitis stimulated a greater leukocyte response (P<0.001). The effect of systemic and other organ infections on breastmilk leukocyte levels was in agreement with the observed effect of mastitis of one breast on breastmilk leukocyte levels of the other (mastitis-free) breast. A small increase in breastmilk leukocyte levels was also observed when only the infant had an infection, while the mother was asymptomatic (P=0.046). No difference was seen between pre- and post-infection baseline leukocyte levels (P=0.48). Importantly, the leukocyte response to infection and recovery, as well as transition from colostrum to transitional breastmilk was rapid. In contrast to leukocytes, breastmilk total cell and viable cell levels did not significantly vary by infection status or breastmilk type (Table 3). However, the number of total cells significantly increased (P=0.002) with lactation stage (Table 2). The latter did not significantly influence % cell viability (P=0.09) (Table 2). Breastmilk leukocyte levels did not significantly change after week 1 postpartum as long as the dyad was healthy (P=0.36). Leukocytes in breastmilk from women with mastitis contained a number of different immune cells such as monocytes, macrophages, dendritic cells, T helper cells, cytotoxic T cells, natural killer cells, and a small population of B lymphocytes. Infection-stimulated leukocytes contained activated cell subsets that responded to viral antigens and PHA in culture with increased proliferation rate and altered expression of IL-6, IL-17A, IFN-γ and TNF-α.

### Infections of the Mother/Infant Dyad Stimulate a Humoral Immune Response in Breastmilk

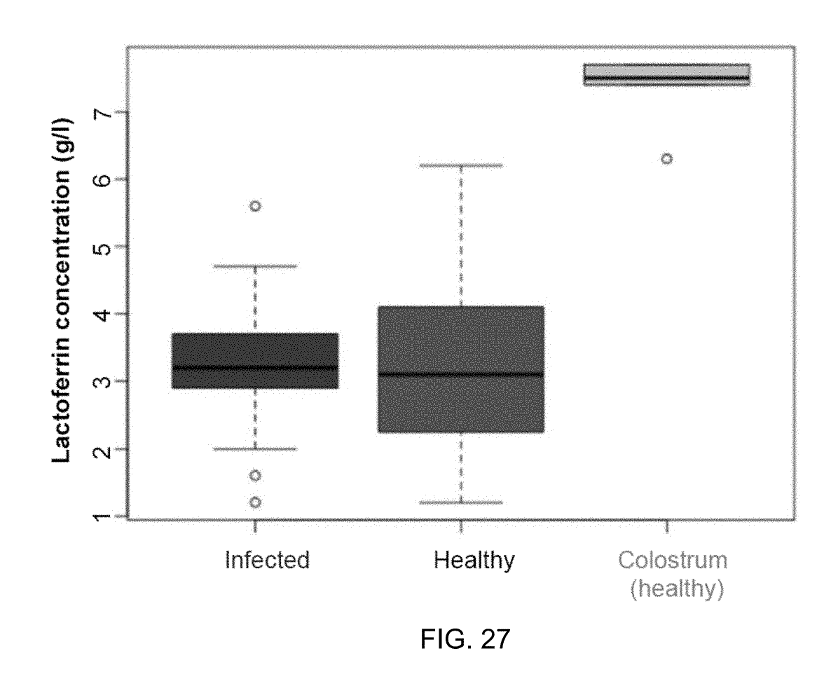

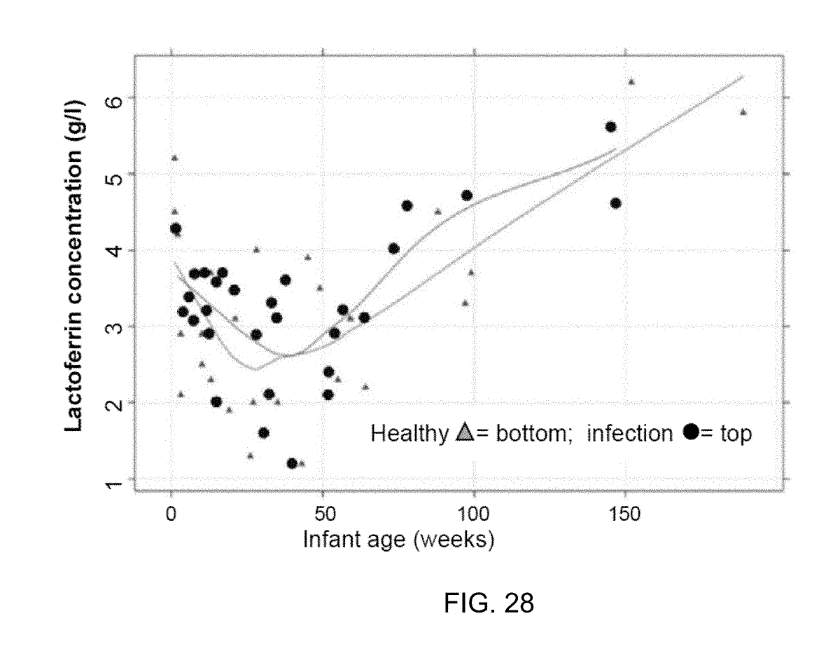

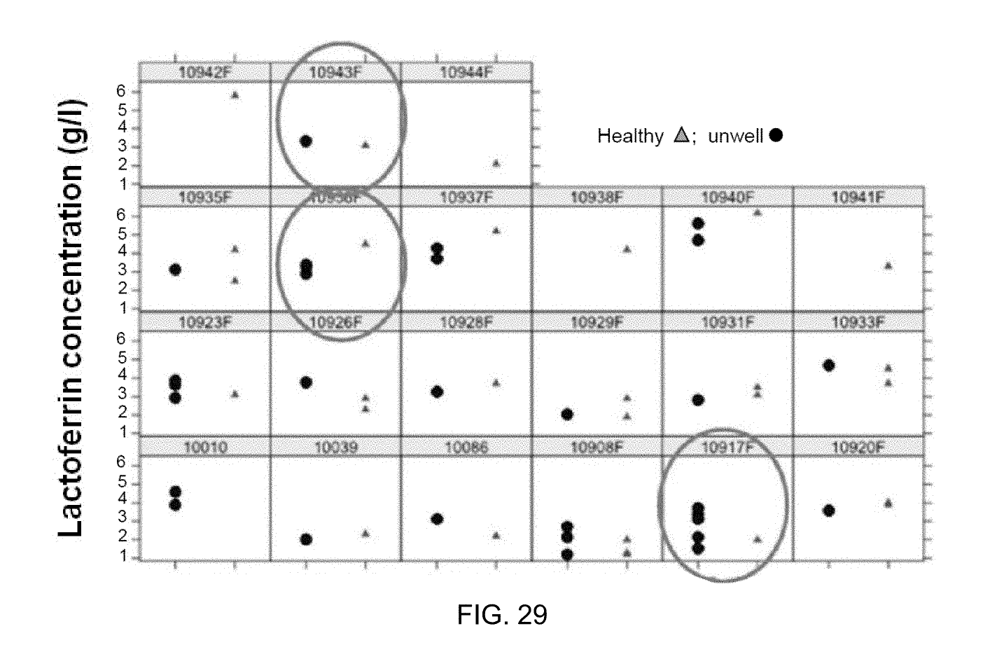

In addition to breastmilk the above-described leukocyte response to maternal/infant infection, a often significant humoral immune response was also observed. sIgA was higher in colostrum compared to mature breastmilk from healthy dyads (P<0.001) (FIGS. 20 to 29; Tables 2 and 3). In mature breastmilk, sIgA concentration increased only during infection of the mother and/or the infant (P=0.034) (FIGS. 20 to 29; Tables 2 and 3), and this increase was stronger in organ-specific infections (Table 4). IgG concentration was generally low (2.8-22.9 μg/mL) (Table 2), with no marked difference between colostrum and mature breastmilk from healthy dyads (P=0.71), and marginally increased with maternal or infant infection (P=0.048) (FIGS. 20 to 29; Tables 2 and 3). No difference was seen between pre- and post-infection baseline sIgA and IgG levels (P=0.37 and P=0.66, respectively). In few subjects, sIgA/IgG concentration was higher in the post-recovery sample, suggesting a potentially delayed response to infection. Infant age had a profound effect on breastmilk sIgA (P<0.001), IgG (P=0.045) and lactoferrin (P=0.008) concentrations (Table 2). In the dataset of healthy dyads, an initial sIgA decrease from colostrum to mature breastmilk up to around week 25 and a plateau until week 50 was followed by an increase in later lactation. IgG concentration was constant for the first 60 weeks postpartum, but increased in later lactation (Table 2). Lactoferrin concentration initially decreased up to around week 25 and then increased as lactation progressed (Table 2). Involution seemed to influence the biochemical and total cellular, but not the leukocyte levels of breastmilk, with marked increases in these components (Table 2).

### Breastmilk Immune Response Differs Between Infection Types

Breastmilk leukocyte levels were significantly higher for all infection types compared to the healthy baseline, with the weakest response seen for infant infections (P=0.046), and the strongest response for breast infections (P<0.001), particularly mastitis (Table 4). A decrease in % cell viability with infection was observed only for maternal colds (P=0.025). Total breastmilk cell levels increased during breast-related infections, being associated with a stronger leukocyte response in mastitis compared to less severe breast infections (Table 4). Principal component analysis (PCA) demonstrated distinctive response patterns for specific sample types. Mastitis (N=5) clustered separately from other infections, being strongly associated with breastmilk leukocyte levels. Weaning (N=1) and menstruation (N=1) were separate from the rest of the healthy dataset. Colostrum also tended to cluster differently from the healthy dataset.

Non-Exclusive Breastfeeding is Associated with Lower Baseline Breastmilk Leukocyte Levels

Breastfeeding status significantly related to breastmilk leukocyte levels. Under healthy conditions, the baseline breastmilk leukocyte levels was lower in non-exclusively breastfed infants (P=0.024), the majority of whom received breastmilk with no detectable leukocytes. The infection response in both exclusive and non-exclusive breastfeeding dyads was such that similar leukocyte levels were seen during infection.

In addition to the description of the results provided above and in the Figures, the results obtained by way of the above-described cellular and biochemical analysis are summarised in Tables 1 to 5 below.

TABLE 1

| | | | | | | | | | | | Total viable | | |
|---|---|---|---|---|---|---|---|---|---|---|---|---|---|
| | | Lactation stage | | | Exclusive | IgA* | IgG* | IgM* | Lact* | ICs | cells/ml | Cell viability | Viable ICs/ml |
| Dyad | Date | (weeks) | Infection status | Milk status | breast feeding | (μg/ml) | (μg/ml) | (μg/ml) | (g/l) | (%) | of milk# | (%) | of milk |
| D 1 | 17 Sep. 2010 | Colostrum | Colostrum | Colostrum | yes | 1428 | 7.3 | 16.2 | 7.5 | NA | 2,250,000 | 96.6 | NA |
| | 1 Oct. 2010 | 3 | healthy | transition milk | yes | 131 | 2.8 | 8.3 | 2.9 | 0 | 113,492 | 97.3 | 0 |
| | 13 Oct. 2011 | 15 | baby mild cold | mature milk | yes | 652 | 4.8 | 12.4 | 2.0 | 1.11 | 265,000 | 96.4 | 2,942 |
| | 11 Feb. 2011 | 19 | healthy | mature milk | yes | 257 | 4.6 | 6.5 | 1.9 | 0 | 229,729 | 100.0 | 0 |
| D 2 | 15/10 | Colostrum | Colostrum | Colostrum | yes | 2178 | 6.9 | 19.1 | 7.7 | 13.2 | 243,750 | 100.0 | 32,175 |
| | 25 Oct. 2010 | 2 | healthy | transition milk | yes | 670 | 9.7 | 14.1 | 4.2 | 0.12 | 883,333 | 97.2 | 1,060 |
| | 6 Dec. 2010 | 7 | sore breast | mature milk | yes | 36 | 10.0 | 8.3 | 3.1 | 0.72 | 333,333 | 96.8 | 2,400 |
| | 27 Dec. 2010 | 10 | healthy | mature milk | yes | 534 | 6.4 | 10.6 | 2.5 | 0.07 | 228,395 | 100.0 | 160 |
| D 3 | 5 Nov. 2010 | Colostrum | Colostrum | Colostrum | yes | 2136 | 12.2 | 24.8 | 7.7 | 39.6 | 1,980,000 | 94.3 | 784,080 |
| | 9 Nov. 2011 | 1 (day 7) | healthy | transition milk | yes | 1096 | 6.6 | 15.4 | 4.5 | 1.65 | 200,758 | 97.2 | 3,313 |
| | 9 Dec. 2010 | 6 | Cold, sore | mature milk | yes | 848 | 6.6 | 9.3 | 3.4 | 22.1 | 50,000 | 93.3 | 11,050 |
| | | | throat | | | | | | | | | | |
| | 13 Jan. 2011 | 11 | blocked duct | mature milk | yes | 594 | 9.0 | 4.5 | 2.9 | 12.5 | 265,000 | 100.0 | 33,125 |
| | 18 Jan. 2011 | 12 | mastitis | mature milk | yes | 696 | 11.7 | 7.8 | 3.2 | 64.9 | 696,070 | 91.0 | 451,749 |
| D 4 | 1 Oct. 2010 | 4 | cold | mature milk | yes | 417 | 13.0 | 30.0 | 3.2 | 82.3 | 194,545 | 87.7 | 160,111 |
| | 29 Nov. 2010 | 13 | healthy | mature milk | yes | 313 | 7.4 | 11.6 | 3.7 | 1.52 | 40,000 | 96.3 | 608 |
| D 5 | 5 Oct. 2010 | 15 | cold | mature milk | yes | 1711 | 10.1 | 11.1 | 3.6 | 33.9 | 321,918 | 96.9 | 109,130 |
| | 22 Oct. 2010 | 17 | mother gastro | mature milk | yes | 1312 | 9.8 | 15.4 | 3.7 | 1.36 | 159,091 | 99 | 2,164 |
| | 17 Nov. 2010 | 21 | minor cold | mature milk | yes | 1049 | 8.7 | 10.1 | 3.5 | 3.19 | 115,278 | 97.6 | 3,677 |
| | 18 Nov. 2010 | 21 | healthy | mature milk | yes | 446 | 6.7 | 9.5 | 3.1 | 0.41 | 250,000 | 96.8 | 1,025 |
| | 10 Jan. 2011 | 28 | cold | mature milk | no | 1509 | 14.4 | 13.2 | 2.9 | 15.1 | 140,000 | 93.3 | 21,140 |
| D 6 | 11 Sep. 2010 | 10 | healthy | mature milk | yes | 1276 | 12.4 | 14.9 | 2.9 | 0.45 | 255,769 | 96.3 | 1,151 |
| | 17 Sep. 2010 | 11 | cold | mature milk | yes | 1401 | 10.7 | 19.8 | 3.7 | 28.8 | 676,191 | 94.0 | 194,743 |
| | 1 Oct. 2010 | 13 | healthy | mature milk | yes | 960 | 10.8 | 9.8 | 2.3 | 0.06 | 588,542 | 100.0 | 353 |
| D 7 | 17 Sep. 2010 | 26 | healthy | mature milk | yes | 496 | 4.0 | 11.1 | 1.3 | 0 | 433,333 | 98.1 | 0 |
| | 27 Oct. 2010 | 32 | baby fever | mature milk | yes | 611 | 7.4 | 14.4 | 2.1 | 1.08 | 98,633 | 97.1 | 1,065 |
| | 17 Nov. 2010 | 35 | healthy | mature milk | yes | 638 | 7.4 | 4.2 | 2.0 | 0.07 | 252,500 | 98.1 | 177 |
| | 27 Dec. 2010 | 40 | sore breast | mature milk | yes | 714 | 8.8 | 19.3 | 1.2 | shift | 437,500 | 100.0 | NA |
| | 18 Jan. 2011 | 43 | healthy | mature milk | no | 401 | 5.0 | 8.8 | 1.2 | 0.1 | 706,667 | 98.1 | 707 |
| | 21 Mar.2011 | 52 | mastitis | mature milk | no | 946 | 25.9 | 18.2 | 2.4 | 71.7 | 1,059,740 | 96.2 | 759,834 |
| D 8 | 17 Sep. 2010 | 28 | healthy | mature milk | no | 1350 | 16.4 | 23.7 | 4.0 | 0 | 183,871 | 93.4 | 0 |
| | 25 Nov. 2010 | 38 | cold | mature milk | no | 789 | 7.6 | 14.4 | 3.6 | 3 | 1,000,000 | 98.8 | 30,000 |
| | 18 Jan. 2011 | 45 | healthy | mature milk | no | 1044 | 16.1 | 23.3 | 3.9 | 0.08 | 1,066,667 | 98.5 | 853 |
| D 9 | 14 Oct. 2010 | 27 | healthy | mature milk | no | 638 | 6.2 | 14.7 | 2.0 | 1.09 | 97,500 | 99.2 | 1,063 |
| | 2 Nov. 2010 | 30 | cold | mature milk | no | 696 | 5.6 | 12.6 | 1.6 | 32.9 | 56,190 | 100.0 | 18,487 |
| | 23 Nov. 2010 | 33 | mastitis | mature milk | no | 1230 | 14.2 | 21.8 | 3.3 | 93.6 | 504,951 | 96.2 | 472,634 |
| | left | | | | | | | | | | | | |
| | 23 Nov. 2010 | 33 | ok | mature milk | no | 831 | 6.6 | 16.0 | 2.1 | 12.9 | 37,000 | 94.9 | 4,773 |
| | right | | | | | | | | | | | | |
| | 8 Dec. 2010 | 35 | recovering | mature milk | no | 1312 | 8.8 | 20.6 | 3.1 | 43.5 | 58,696 | 90.0 | 25,533 |
| | left | | mastitis | | | | | | | | | | |
| D 10 | 9 Nov. 2010 | 49 | healthy | mature milk | no | 137 | 7.0 | 5.2 | 3.5 | 0 | 358,974 | 100.0 | 0 |
| | 16 Dec. 2010 | 54 | measles | mature milk | no | 173 | 6.9 | 5.9 | 2.9 | 2.13 | 60,714 | 97.1 | 1,293 |
| | 18 Jan. 2011 | 59 | healthy | mature milk | no | 244 | 6.0 | 4.9 | 3.1 | 0 | 102,778 | 97.4 | 0 |
| D 11 | 11 Jan. 2011 | 64 | recovering | mature milk | no | 1179 | 8.2 | 6.8 | 3.1 | 2.61 | 174,603 | 99.1 | 4,557 |
| | | | vaginal thrush | | | | | | | | | | |
| | 13 Jan. 2011 | 64 | healthy | mature milk | no | 743 | 4.0 | 2.9 | 2.2 | 0 | 1,075,000 | 97.8 | 0 |
| D 12 | 4 Nov. 2010 | 88 | healthy | mature milk | no | 1243 | 7.4 | 8.1 | 4.5 | 0.06 | 480,769 | 97.1 | 288 |
| | 10 Jan. 2011 | 98 | gastro | mature milk | no | 1657 | 9.4 | 10.5 | 4.7 | 4.27 | 1,166,667 | 98.6 | 49,817 |
| | 18 Jan. 2011 | 99 | healthy | mature milk | no | 1240 | 12.7 | 13.0 | 3.7 | 0 | 3,357,143 | 98.3 | 0 |
| D 13 | 6 Dec. 2010 | Colostrum | Colostrum | Colostrum | yes | 2008 | 7.1 | 56.1 | 7.4 | 70.4 | 110,000 | 88.7 | 77,440 |
| | 9 Dec. 2010 | 1 (day 6) | healthy | transition milk | yes | 1044 | 7.0 | 24.8 | 5.2 | 0.59 | 406,667 | 95.3 | 2,399 |
| | 16 Dec. 2010 | 2 (day 13) | cold | transition milk | yes | 922 | 13.0 | 10.2 | 4.3 | 18.8 | 183,333 | 94.3 | 34,467 |
| | 27 Jan. 2011 | 8 | mastitis | mature milk | yes | 1418 | 17.1 | 10.7 | 3.7 | 90.5 | 2,867,383 | 98.0 | 2,594,982 |
| D 14 | 13 Jan. 2011 | Colostrum | Colostrum | Colostrum | yes | 1883 | 5.3 | 37.0 | 6.3 | 38.4 | 577,778 | 96.3 | 221,867 |
| | 20 Jan. 2011 | 2 | healthy | transition milk | yes | 812 | 6.8 | 29.8 | 4.2 | 0.96 | 359,375 | 100.0 | 3,450 |
| D 15 | 13 Jan. 2011 | 97 | healthy | mature milk | no | 976 | 4.6 | 7.1 | 3.3 | 0 | 633,333 | 100.0 | 0 |
| D 16 | 20 Oct. 2010 | 52 | cold | mature milk | no | 506 | 2.3 | 10.8 | 2.1 | 2.87 | 102,326 | 97.8 | 2,937 |
| | 9 Nov. 2010 | 55 | healthy | mature milk | no | 621 | 4.0 | 13.0 | 2.3 | 0 | 75,926 | 100 | 0 |
| D 17 | 10 Jan. 2011 | 74 | cold | mature milk | no | 2002 | 8.5 | 31.1 | 4.0 | 4.74 | 420,000 | 97.7 | 19,908 |
| | 11 Feb. 2011 | 78 | eye infection | mature milk | no | 2184 | 11.0 | 48.8 | 4.6 | 12.5 | 1,708,333 | 97.6 | 213,542 |
| D 18 | 10 Jan. 2011 | 146 | cold | mature milk | no | 1906 | 13.4 | 23.5 | 5.6 | 6.36 | 49,166 | 96.7 | 3,127 |
| | 19 Jan. 2011 | 147 | ear infection | mature milk | no | 1888 | 11.4 | 22.1 | 4.6 | 10.8 | 126,786 | 95.9 | 13,693 |
| | 14 Feb. 2011 | 152 | healthy - | weaning milk | no | 1761 | 22.9 | 100.4 | 6.2 | 0 | 2,600,000 | 96.3 | 0 |
| | | | WEANING | | | | | | | | | | |
| D 19F | 19 Jan. 2011 | 189 | healthy - | mature milk | no | 1991 | 17.8 | 23.1 | 5.8 | 0.55 | 2,500,000 | 100.0 | 13,750 |
| | | | PERIODS | | | | | | | | | | |
| D 20 | 27 Jan. 2011 | 57 | mastitis | mature milk | no | 722 | 12.7 | 5.9 | 3.2 | 58.9 | 400,000 | 96.3 | 235,600 |
| | 9 Sep. 2011 | 59 | healthy | mature milk | no | 485 | 10.4 | 4.3 | 3.1 | 0 | 177,778 | 96.9 | 0 |
| D 21 | 15 Mar. 2011 | 3 | healthy | transition milk | yes | 275 | 5.3 | 8.2 | 2.1 | 0.67 | 203,704 | 91.7 | 1,365 |

*Average

TABLE 2
Total cell, leukocyte, immunoglobulin (sIgA, IgG, IgM) and lactoferrin contents of human breastmilk
at different stages of lactation, and responses to infections of the mother/infant dyad.

| | | | Transitional | Months | Months | Months | Months | | Late | Involution |
|---|---|---|---|---|---|---|---|---|---|---|
| Breastmilk | Health | Colostrum^{a} | milk^{b} | 1-3 | 4-6 | 7-9 | 10-12 | Year 2 | lactation^{c} | milk^{d} |
| component | status | n = 5h | n = 6h, 1i | n = 2h, 7i | n = 4h, 4i | n = 4h, 6i | n = 2h, 2i | n = 6h, 7i | n = 3h, 3i | n = 1 |
| Total cells^{e}/ | Healthy | 110,000- | 113,492- | 228,395- | 40,000- | 97,500- | 706,667- | 75,926- | 633,333- | 2,600,000 |
| mL milk | | 2,250,000 | 883,333 | 255,769 | 588,542 | 433,333 | 1,066,667 | 1,075,000 | 3,357,143 | |
| | Infection | — | 183,333 | 50,000- | 115,278- | 37,000- | 437,500- | 60,714- | 49,166- | — |
| | | | | 2,867,383 | 321,918 | 504,951 | 1,000,000 | 1,708,333 | 1,166,667 | |
| % Leukocytes | Healthy | 13.2-70.4 | 0.0-1.65 | 0.07-0.45 | 0.0-1.52 | 0.0-1.09 | 0.08-0.1 | 0.0-0.06 | 0.0-0.55 | 0.0 |
| | Infection | — | 18.8 | 0.72-90.5 | 1.1-33.9 | 1.08-93.6 | >3 | 2.13-71.7 | 4.27-10.8 | — |
| Leukocytes^{e}/ | Healthy | 32,175- | 0-3,450 | 160- | 0-1,025 | 0-1,063 | 707-853 | 0-288 | 0-13,750 | 0 |
| mL milk | | 784,080 | | 1,151 | | | | | | |
| | Infection | — | 34,467 | 2,400- | 2,164- | 1,065- | >30,000 | 1,293- | 3,127- | — |
| | | | | 2,594,982 | 109,130 | 472,634 | | 59,834 | 49,817 | |
| sIgA (μg/mL) | Healthy | 1428-2178 | 131-1096 | 534-1276 | 257-960 | 496-1350 | 401-1044 | 137-1243 | 976-1991 | 1761 |
| | Infection | — | 922 | 36-1418 | 652-1711 | 611-1509 | 714-789 | 173-2002 | 1657-1906 | — |
| IgG (μg/mL) | Healthy | 5.3-12.2 | 2.8-9.7 | 6.4-12.4 | 4.6-10.8 | 4.0-16.4 | 5.0-16.1 | 4.0-10.4 | 4.6-17.8 | 22.9 |
| | Infection | — | 13.0 | 6.6-17.1 | 4.8-10.1 | 5.6-14.4 | 7.6-8.8 | 2.3-25.9 | 9.4-13.4 | — |
| IgM (μg/mL) | Healthy | 16.2-56.1 | 8.2-29.8 | 10.6-14.9 | 6.5-11.6 | 4.2-23.7 | 8.8-23.3 | 2.9-13.0 | 7.1-23.1 | 100.4 |
| | Infection | — | 10.2 | 4.5-19.8 | 10.1-15.4 | 12.6-21.8 | 14.4-19.3 | 5.9-31.1 | 10.5-23.5 | — |
| Lactoferrin | Healthy | 6.3-7.7 | 2.1-5.2 | 2.5-2.9 | 1.9-3.7 | 1.3-4.0 | 1.2-3.9 | 2.3-4.5 | 3.3-5.8 | 6.2 |
| (g/L) | Infection | — | 4.3 | 2.9-3.7 | 2.0-3.7 | 1.6-3.3 | 1.2-3.6 | 2.1-4.6 | 4.6-5.6 | — |

^{a}Colostrum was defined as breastmilk collected between days 0-4 postpartum;
^{b}Transitional milk was defined as breastmilk collected between day 5 and week 3 postpartum;
^{c}Late lactation was defined as the period between months 25 and 48 postpartum;
^{d}Involution milk was collected 5 days after baby weaned off breastmilk (month 38 postpartum);
^{e}Total cells/mL and leukocytes/mL refer to total viable cells/mL and viable leukocytes/mL, respectively.
(h = healthy and i = infection)

TABLE 3
Statistical comparison of the levels of measured variables between milk
samples grouped by type (colostrum versus mature breastmilk from healthy
mother/infant dyads) or health status of the mother/infant dyad (healthy
versus under infection), taking into account individual differences.

| | | Healthy | Under infection | | | |
|---|---|---|---|---|---|---|
| | | (mature milk) | (mature milk) | | Colostrum | |
| Response | Transform | Value | Diff | P value | Diff | P value |
| total cell content | logₑ | 12.8 | −0.3 | 0.271 | 0.5 | 0.326 |
| (/mL milk) | | | | | | |
| viable cell content | logₑ | 12.8 | −0.3 | 0.253 | 0.5 | 0.353 |
| (/mL milk) | | | | | | |
| leukocyte content^{a} | logₑ(x + 0.5) | 3.8 | 5.9 | <0.001 | 7.2 | <0.001 |
| (/mL milk) | | | | | | |
| % total cell viability | none | 97.8 | −1.5 | 0.043 | −2.6 | 0.056 |
| (of total cells) | | | | | | |
| % leukocytes^{a} | logₑ(x + 0.5) | −0.25 | 2.5 | <0.001 | 3.6 | <0.001 |
| (of total cells) | | | | | | |
| sIgA (μg/mL) | none | 860.7 | 169.9 | 0.034 | 1288.5 | <0.001 |
| IgG (μg/mL) | logₑ | 2.01 | 0.22 | 0.048 | 0.07 | 0.710 |
| IgM (μg/mL) | logₑ | 2.49 | 0.064 | 0.606 | 0.864 | <0.001 |
| Lactoferrin (g/L) | none | 3.41 | −0.08 | 0.658 | 3.88 | <0.001 |

^{a}For leukocyte content and percentage, the data was transformed using the additive constant 0.5 for both the square root and the log transformations due to the zeroes in the data (Yamamura K. 1999)
P values compare each group with the “Healthy (mature milk)” group.

TABLE 4
Effects of different types of infection on breastmilk cellular and biochemical composition.

| | | | | | | | | Other | |
|---|---|---|---|---|---|---|---|---|---|
| | Healthy | Infant only | | Breast-related | | Cold | | infections | |
| Response | Value | Diff | P value | Diff | P value | Diff | P value | Diff | P value |
| total cell content | 12.8 | −0.9 | 0.127 | 0.6 | 0.133 | −0.6 | 0.094 | −0.4 | 0.348 |
| (/mL milk) (logₑ) | | | | | | | | | |
| viable cell | 12.8 | −0.9 | 0.123 | 0.6 | 0.143 | −0.6 | 0.085 | −0.4 | 0.345 |
| content | | | | | | | | | |
| (/mL milk) (logₑ) | | | | | | | | | |
| leukocyte | 3.7 | 4.3 | 0.046 | 6.7 | <0.001 | 6.1 | <0.001 | 5.9 | 0.0004 |
| content^{a} | | | | | | | | | |
| (/mL milk) | | | | | | | | | |
| (logₑ(x + 0.5)) | | | | | | | | | |
| % total cell | 97.8 | −0.9 | 0.571 | −1.7 | 0.093 | −2.1 | 0.025 | −0.2 | 0.834 |
| viability | | | | | | | | | |
| (of total cells) | | | | | | | | | |
| % leukocytes^{a} | −0.3 | 1.1 | 0.064 | 3.2 | <0.001 | 2.8 | <0.001 | 2.1 | <0.001 |
| (of total cells) | | | | | | | | | |
| (logₑ(x + 0.5)) | | | | | | | | | |
| sIgA | 858 | 88 | 0.632 | 174 | 0.155 | 144 | 0.197 | 302 | 0.042 |
| IgG (logₑ) | 2.02 | 0.07 | 0.738 | 0.58 | 0.0003 | 0.04 | 0.758 | 0.12 | 0.481 |
| IgM (logₑ) | 2.49 | 0.21 | 0.474 | 0.05 | 0.796 | 0.02 | 0.913 | 0.16 | 0.493 |
| lactoferrin | 3.4 | −0.2 | 0.638 | −0.1 | 0.787 | −0.1 | 0.654 | 0.1 | 0.766 |

Groups include: infant only infection (N = 3), breast-related infection (N = 9), cold (N = 12), other organ-specific infections (eye, ear, vaginal, urinary tract and gastrointestinal infections; N = 6), and no infection/healthy (N = 28).
^{a}For leukocyte content and percentage, the data was transformed using the additive constant 0.5 for both the square root and the log transformations due to the zeroes obtained (Yamamura K. 1999)
P values compare infection groups with the “Healthy” group.

TABLE 5

| | Dyad | | | | | | | |
|---|---|---|---|---|---|---|---|---|
| | D 22 | D 23 | D 24 | D 25 | D 26 | D 27 | D 28 | D 29 |
| | Infection status | | | | | | | |
| | | mother-mastitis- | | | mother | | | |
| | | recurring | | | recovering | | | |
| | infant-only | nipple/vaginal | mother/baby | infant-only | from | mother/baby | mother | mother/baby |
| | fever | thrush | healthy | cold | mastitis | healthy | cold | healthy |
| CD45+ [%] | 9.78 | 44.2 | 1.82 | 2.27 | 30.86 | 1.84 | 3.18 | 0.3 |
| CD45+/CD45RO+ [%] | 8.93 | | | | | | | 0.28 |
| CD45+/CD45RO− [%] | 0.85 | | | | | | | 0.02 |
| CD45+/CD14+ [%] | 4.17 | | | | 20.06 | | | 0.15 |
| CD45+/CD14− [%] | 5.57 | | | | 10.8 | | | 0.15 |
| CD45−/CD14+high [%] | 18.7 | | | | 60.8 | | | 89.3 |
| CD45−/CD14+low [%] | 70.2 | | | | | | | |
| total CD14+ [%] | 93.07 | | | | 80.86 | | | 89.45 |
| CD45+/CD15+high[%] | 1.01 | | 0.37 | 0.75 | 6.06 | | | |
| CD45+/CD15− [%] | 8.72 | | 1.45 | 1.52 | 24.8 | | | |
| CD45−/CD15+high [%] | 1.75 | | | 1.99 | 2.97 | | | |
| CD45−/CD15+low [%] | 8.79 | | | | | | | |
| CD15+high total [%] | 2.76 | | | | | | | |
| CD15+total [%] | 11.55 | | 3.32 | 2.73 | 9.03 | | | |
| CD45+/HLA−DR+ [%] | 5.88 | | | | | | | |
| CD45+/HLA−DR−low [%] | 3.86 | | | | | | | |
| CD45−/HLA−DR+ [%] | 67.3 | | | | | | | |
| HLA+ total [%] | 73.18 | | | | | | | |
| CD45+/CD3+/CD4+ | 2.014 | 14.01 | 0.41 | 0.203619 | 5.9444 | 0.34 | 0.8 | 0.0486 |
| (t-helper) [%] | | | | | | | | |
| CD45+/CD3+ [%] | 2.61 | 18.1 | 0.79 | 0.35 | 8.96 | 0.61 | 1.61 | 0.094 |
| total CD3+ | 2.61 | 18.1 | 0.79 | 0.35 | 8.96 | 0.61 | 1.61 | 0.0936 |
| (total T-cells) [%] | | | | | | | | |
| CD45+/CD3− [%] | 7.17 | 26.1 | 1.03 | 1.92 | 21.84 | 1.23 | 1.57 | 0.2064 |
| CD45+/CD4+ [%] | 4.14 | 22.8 | 0.45 | 1.05 | 7.71 | 0.47 | 0.84 | 0.064 |
| total CD4+ [%] | 4.14 | 22.8 | 0.45 | 1.048 | 7.71 | 0.75 | 1.03 | 0.064 |
| CD45+/CD4− [%] | 5.64 | 21.4 | 1.37 | 1.22 | 23.09 | 1.37 | 2.34 | 0.236 |
| CD45+/CD3+/CD8+ | 0.908 | 4.95 | 0.195 | 0.088757 | 2.68 | 0.25 | 0.86178 | 0.0405 |
| (t-cytotoxic) [%] | | | | | | | | |
| CD45+/CD8+ [%] | 0.85 | 6.1 | 0.24 | 0.79 | 4.34 | 0.27 | 0.98 | 0.091 |
| total CD8+ [%] | 0.85 | 15.3 | 0.24 | 0.79 | 4.33664 | 0.27 | 1.07 | 0.0906 |
| CD45+/CD8− [%] | 8.93 | 38.1 | 1.58 | 1.48 | 26.52 | 1.57 | 2.2 | 0.209 |
| CD45+/CD3+/CD56+ | 0.091 | 0.703 | 0.034 | 0.066511 | 0.1848 | 0.018 | 0.167 | 0.02211 |
| (NK-T-cells) [%] | | | | | | | | |
| CD45+/CD3−/CD56+ | 0.3 | 0.87 | 0.047 | 0.71051 | 1.16732 | 0.065 | 0.789 | 0.00633 |
| (other NK-cells) [%] | | | | | | | | |
| CD45+/CD56+ | 0.391 | 1.573 | 0.081 | 0.777021 | 1.35212 | 0.083 | 0.956 | 0.02844 |
| (total NK-cells) [%] | | | | | | | | |
| total CD56+ [%] | 0.391 | 16.9 | 0.081 | 0.777021 | 1.35212 | 0.083 | 0.956 | 0.02844 |
| CD45+/CD56− [%] | 9.389 | 42.1 | 1.739 | 1.492979 | 29.50788 | 1.757 | 2.224 | 0.27156 |
| CD45+/CD3+/CD16+ | 0.17 | | | | | | | |
| (NK-T-cells) [%] | | | | | | | | |
| CD45+/CD3−/CD16+ [%] | 3.11 | | | | | | | |
| CD45+/CD16+ [%] | 4.05 | | | | | | | |
| CD45+/CD16− [%] | 5.87 | | | | | | | |
| CD45−/CD16+ [%] | 5.86 | | | | | | | |
| CD45+/CD19+ [%] | 5.11 | | | | | | | |
| CD45+/CD19− [%] | 4.79 | | | | | | | 0.019 |
| total CD19+ [%] | 5.11 | | | | | | | 0.019 |
| CD45+/CD25+ [%] | 8.1 | | | | | | | |
| CD45+/CD25− [%] | 1.76 | | | | | | | |
| CD45−/CD25+ [%] | 0.13 | | | | | | | |
| total CD25+ [%] | 8.23 | | | | | | | |

Thus, while there has been described what are believed to be the embodiments of the invention, those skilled in the art will recognize that other and further modifications may be made thereto without departing from the spirit of the invention, and it is intended to claim all such changes and modifications as falling within the scope of the invention.

1-8. (canceled)

9. A method of treatment comprising:
(a) measuring at least one immunological marker in a breastmilk sample from a breastfeeding mother, wherein the at least one immunological marker comprises a cellular and/or a humoral immunological marker;
(b) detecting an elevated measure of the at least one immunological marker by comparing the measurement of the at least one marker to a reference measure of the marker in breastmilk of a healthy subject;
(c) diagnosing an infection in the mother and/or an infant breastfed by the breastfeeding mother from the elevated measure of the at least one immunological marker;
(d) treating the infection with an antibiotic;
(e) measuring the at least one immunological marker in a breastmilk sample after the treating.

10. The method of claim 9 that further comprises:
(f) detecting an elevated measure of the at least one immunological marker in step (e) by comparing the measurement of the at least one marker to a reference measure of the marker in breastmilk of a healthy subject; and
(g) treating the infection with a different antibiotic from the antibiotic of step (d).

11. The method according to claim 10, wherein the cellular and/or humoral immunological marker comprises interleukin-2 (IL-2), interleukin-4 (IL-4), interleukin-6 (IL-6), interleukin-10 (IL-10), interleukin-17a (IL-17a), or interferon-gamma (IFNγ).

12. The method according to claim 10, wherein the cellular and/or humoral immunological marker comprises immunoglobulin M (IgM).

13. The method according to claim 10, wherein the cellular and/or humoral immunological marker comprises live CD45-positive immune cells.

14. The method according to claim 13, wherein the CD45-positive cells comprise one or more populations of T-cells.

15. The method according to claim 9, that comprises performing at least the measuring and detecting steps with a breastmilk sample from each breast.

16. A method of evaluating donated samples of breastmilk comprising:
(a) measuring at least one immunological marker in breastmilk samples from at least one lactating mother, wherein the at least one immunological marker is selected from an immune cell (IC), a cytokine, and an immunoglobulin;
(b) comparing the measurement of the at least one marker to a reference measure of the marker in breastmilk of a healthy subject;
(c) (1) discarding, as unsuitable for heterologous consumption, breastmilk samples with an elevated measurement of the last least one immunological marker, and (2) selecting for banking or feeding to an infant that is not the child of the lactating mother (heterologous recipient), breastmilk samples free of the elevated measurement of the immunological marker.

17. The method according to claim 16, wherein the immunological marker comprises a cytokine.

18. The method according to claim 17, wherein the cytokine comprises interleukin-2 (IL-2), interleukin-4 (IL-4), interleukin-6 (IL-6), interleukin-10 (IL-10), interleukin-17a (IL-17a), or interferon-gamma (IFNγ).

19. The method according to claim 16, wherein the immunological marker comprises a live CD45-positive immune cell.

20. The method according to claim 19, wherein the CD45-positive cell comprises one or more populations of T-cells.

21. The method according to claim 16, wherein the immunological marker comprises an immunoglobulin.

22. The method according to claim 21, wherein the immunoglobulin comprises immunoglobulin M (IgM).
